(19) United States
(12) Patent Application Publication
(10) Pub. No.: US 2022/0347276 A1
(43) Pub. Date: Nov. 3, 2022

(54) ANC80 ENCODING SPHINGOLIPID-METABOLIZING PROTEINS FOR MITIGATING DISEASE-INDUCED TISSUE DAMAGE

(71) Applicant: ICAHN SCHOOL OF MEDICINE AT MOUNT SINAI, New York, NY (US)

(72) Inventors: Efrat ELIYAHU, New York, NY (US); Adam VINCEK, New York, NY (US); Anthony FARGNOLI, New York, NY (US); Michael KATZ, New York, NY (US)

(21) Appl. No.: 17/753,708

(22) PCT Filed: Sep. 11, 2019
(86) PCT No.: PCT/US19/50634

§ 371 Date: Mar. 11, 2022

(51) Int. Cl. A61K 38/50; A61K 38/45; A61K 38/17

(52) U.S. Cl. CPC A61K 38/50; A61K 38/45; A61K 38/177

(57) ABSTRACT

The present disclosure relates generally to the use of sphingolipid-metabolizing proteins to mitigate or minimize tissue damage resulting from injury or from disease, for example, pulmonary arterial hypertension (PAH) when the sphingolipid-metabolizing protein is delivered via expression from an Anc80 vector.

### SEQUENCE LISTING

The instant application contains a Sequence Listing, created on Dec. 18, 2018; the file, in ASCII format, is designated 3710053WO_SequenceListing_ST25.txt and is 39.9 kilobytes in size. The file is hereby incorporated by reference in its entirety into the instant application.

### TECHNICAL FIELD

The present disclosure relates generally to the use of sphingolipid-metabolizing proteins to mitigate tissue damage resulting from disease. In pulmonary arterial hypertension, for example, exposure to sphingolipid metabolizing proteins such as acid ceramidase protein expressed from an Anc80 vector inhibits increases in pulmonary vascular resistance and elevation of mean pulmonary artery pressure that lead to pulmonary and cardiac damage and in some cases, cardiac failure.

### BACKGROUND OF THE DISCLOSURE

Pulmonary arterial hypertension (PAH) is a devastating cardiopulmonary disease of the pre-capillary arterial system in the lungs. PAH is a specific type of pulmonary hypertension that is caused by the development of scar tissue in the tiny blood vessels of the lung. This scar tissue blocks the blood flow through the lungs and causes the pressure in those blood vessels to increase. Progressive remodeling of the pulmonary circulation leads to dramatic increases in pulmonary vascular resistance (PVR) and elevated mean pulmonary artery pressure. Normally, the right ventricle outputs blood with ease into low resistance lung anatomy. However, in PAH, this sustained increase in PVR working against normal outflow affects the right ventricle, which must contract with more force to overcome this level of resistance and eventually fails. In the extreme cases, PAH becomes deadly very quickly as right ventricular volume loading can increase greater than 5 times normal, distorting the function of the left ventricle. In this scenario, biventricular dysfunction is noted with rapid decline in cardiac output with death due to pump failure. There is also a high incidence of sudden death due to arrhythmias since stretching of the right ventricle/atria structures triggers deadly conditions.

In PAH, the pulmonary tissue is under a constant cycle of proliferation, clotting, fibrosis, and arterial remodeling. This cycle allows plexiform lesions to develop gradually in the pre-capillary arterial system. These lesions are areas of multiple closed vessel networks that become pathological and invade, destroy neighboring networks. The net effect is a progressive destruction of the majority of pulmonary microcirculation that increases PVR and leads to heart failure.

PAH is typically diagnosed in patients via catheterization and considered positive if mean pulmonary artery pressure (mPAP) is greater than 25 mmHg. Numerous drugs to lower pressure specific to the lung arterioles are given to address the symptom, however does not treat the vascular problem. The disease has 5 distinct groups by etiology, all causing elevation in mPAP: Group 1: Pediatric and or genetic form caused by BMPR2 mutations and others that cause smooth muscle proliferation. Group 2: Secondary to severe left heart failure; post capillary. Largest market since patients with ischemic heart disease often suffer from PAH. Group 3: Due to COPD and other lung disorders which lead to inflammation/debris triggers affecting circulation. Group 4: Thromboembolic: Acute cases from large clots in the pulmonary vasculature. Group 5: Idiopathic.

The standard of care for PAH is a well-developed array of drugs that reduce PVR in the pulmonary arterioles, by acting on 1 of 3 defined pathways: 1) nitric oxide (NO), 2) prostacyclin, and 3) endothelin I/II. The pathways reduce PVR by increasing nitric oxide to relax smooth muscle and dilate vessels, or by interfering with smooth muscle proliferation to prevent closure, directly help blood flow, and maintain patency. These pathways do not ameliorate or interrupt the formation of plexiform lesions. Plexiform lesions are prevalent in >80% of patients post mortem, whereby any drug therapy that was successful in lowering mean PAP for any period of time did not prevent right heart failure and subsequent death. In fact, all drugs are limited in PAH and just focus on pressure reduction, which is controversial. Thus, the use of drugs that alleviate mPAP and treat the cellular mechanisms is a challenge.

What is needed is a therapeutic method that provides long-term expression of a sphingolipid-metabolizing enzyme to inhibit cell death and senescence and initiate survival in cells and tissues damaged by disease such as PAH.

### SUMMARY OF THE DISCLOSURE

A treatment for minimizing cellular/tissue damage resulting from disease, for example PAH, or injury (endothelial, vascular smooth muscle, and pneumocytes), which prevents further deterioration of the tissue, is currently unavailable. Gene therapy works by safely transferring an episomal (i.e. not integrated) DNA instruction for prolonged expression. This therapy, while it may not address the underlying cause of the disease itself, can help minimize the damage to tissues affected by the disease, for example, the poor pulmonary circulation resulting from PAH. Therefore, the present disclosure contemplates administration to the lungs via aerosol or nebulization of a synthetic, ancestral adenovirus, Anc80 that encodes a sphingolipid-metabolizing protein as a novel, robust treatment option for PAH.

The present disclosure therefore, provides a method for minimizing tissue damage resulting from PAH by administration of a sphingolipid metabolizing protein for promoting survival and restoring function of cells or tissue in vitro or in vivo. Administration is by means of a viral vector that encodes the sphingolipid-metabolizing protein; in one embodiment Anc80 that encodes expression of acid ceramidase is administered to a subject in need thereof for the treatment of PAH.

A sphingolipid-metabolizing protein is selected from the group consisting of (1) ceramidase; (2) sphingosine kinase (SPHK); (3) sphingosine-1-phosphate receptor (SIPR); (4) ceramidase kinase (CERK) or a combination of (1), (2), (3), and (4).

In one embodiment, the sphingolipid-metabolizing protein is a ceramidase. In one embodiment the sphingolipid-metabolizing protein is an acid ceramidase. In one embodiment, the sphingolipid-metabolizing protein is a neutral ceramidase. In yet another embodiment, the sphingolipid-metabolizing protein is an alkaline ceramidase. In one embodiment, ceramidase is encoded by a nucleic acid selected from the group consisting of SEQ ID NO: 1, SEQ ID NO: 6, SEQ ID NO: 7, SEQ ID NO: 8, SEQ ID NO: 9, SEQ ID NO: 10, SEQ ID NO: 11 and SEQ ID NO: 12.

In yet another aspect, the disclosure relates to a method in which the vector encoding the expression of sphingolipid-metabolizing protein is Anc80. In one embodiment, the nucleotide sequence of Anc80 that encodes the sphingolipid-metabolizing protein comprises the nucleotide sequence of SEQ ID NO: 20.

In another related aspect, the disclosure relates to a pharmaceutical composition comprising an Anc80 viral vector encoding a sphingolipid-metabolizing protein and a pharmaceutically acceptable carrier.

In yet another related aspect, the disclosure relates to an Anc80 viral vector encoding a sphingolipid-metabolizing protein for use in the treatment of PAH.

In one aspect, the disclosure relates to a method to improve patient outcome in patients with PAH comprising contacting lung cells or tissue with (1) an Anc80 that encodes ceramidase, (2) an ANC80 that encodes sphingosine kinase (SPHK), (3) an ANC80 that encodes sphingosine-1-phosphate receptor (S1PR) (4) an ANC80 that encodes a ceramide kinase (CERK), or any combination of (1), (2), (3) and (4).

Anc80 is a synthetic vector (see Zinn et al. In Silico Reconstruction of the Viral Evolutionary Lineage Yields a Potent Gene Therapy Vector, Cell Reports 12. 1056-1068 (2015), and U.S. Pat. No. 9,695,220; both references are hereby incorporated by reference), contains a nucleotide sequence that encodes acid ceramidase having the oligonucleotide sequence of SEQ ID NO: 1. In one embodiment, the Anc80 encoding AC has the oligonucleotide sequence of SEQ ID NO: 6. In another embodiment, the cells are contacted with Anc80 that encodes sphingosine kinase (SPHK) having the oligonucleotide sequence of SEQ ID NO: 2. In another embodiment, the sphingolipid metabolizing molecule is S1PR and the oligonucleotide encoding it has the sequence SEQ ID NO: 3. In another embodiment, the sphingolipid metabolizing molecule is CERK and the oligonucleotide encoding it has the sequence SEQ ID NO: 19)

In one aspect, the present disclosure relates to a method for treating a subject to mitigate or minimize the tissue damage that results from PAH or other disease or disorder, the method comprising administering to the subject a therapeutically effective dose of an Anc80 viral vector that codes for the expression of a sphingolipid-metabolizing protein. In one embodiment, the sphingolipid-metabolizing protein is selected from the group consisting of (1) a ceramidase; (2) sphingosine kinase (SPHK); (3) sphingosine-1-phosphate receptor (SIPR); (4) ceramidase kinase (CERK) or a combination of (1), (2), (3), and (4). Administration of the sphingolipid-metabolizing protein is via means know to those of skill in the art, for example atomizer or nebulizer.

Compositions comprising any combination of Anc80s that code for the expression of (1) a ceramidase, (2) sphingosine kinase (SPHK), (3) sphingosine-1-phosphate receptor (S1PR) and a (4) CERK are encompassed by the present disclosure.

### BRIEF DESCRIPTION OF THE DRAWINGS

FIG. 1 is a graph showing percent survival for individuals with pulmonary arterial hypertension (PAH)

FIG. 2 is a graph showing that right ventricle (RV) function predicts mortality in PAH.

FIG. 3 shows seven year survival estimates of patients in the REVEAL registry. The REVEAL Registry is a multicenter, observational, U.S.-based study of the clinical course and disease management of PAH.

FIG. 4 shows biodistribution of Anc80 in a rat model. Rats were injected with Anc80 encoding luciferase. 72 hours post injection luciferase activity was assessed using IVIS machine.

FIG. 5 shows severe pulmonary artery hypertension (PAH) in the left pneumonectomy combined with Sugen rat model. In this model right ventricular systolic pressure and mean pulmonary artery pressure significantly increased after 6 weeks.

FIG. 6A-6C shows photomicrographs of hematoxylin and eosin (H&E) staining of lung tissue in PAH. Representative photomicrographs of H&E staining of lung tissue. A Normal lung. B-C Pathological vascular remodeling in PAH rats (pneumonectomy and Sugen). The lung shows concentric medial and intimal thickening (white and black arrows) and severe constricted pulmonary vessels.

FIG. 7 shows hemodynamic data after Anc80 AC intra-tracheal injection. Rats were subjected to PAH induction protocol. On day 0, rats were subjected to baseline MRI, RV and PA catheterization to measure the pressure, and left lung removal. On day 7, pneumonectomized rats were subjected to SU5416 (Su/Pn 10 mg/kg) administration (SC injection). Induced animals demonstrated severely elevated mean PA pressures and developed neointima and smooth muscle hypertrophy. At week 4, PAH induced rats were treated with Anc80 AC (1×10¹¹ genome copies). At week 6 and 8, animals were validated by MRI for heart function and RV and PA catheterization for pressure measurement. Treated animals with AC Anc80 at 8 weeks showed excellent cardiac function (validated by MRI) and normal PA pressures despite PAH disease present. After AC administration cardiac output increased 32%.

FIG. 8 shows hemodynamic data after Anc80 AC intra-tracheal injection. Rats were subjected to PAH induction protocol. On day 0 rats were subjected to baseline MRI, RV and PA catheterization to measure the pressure, and Left lung removal. On day 7, pneumonectomized rats were subjected to SU5416 (Su/Pn 10 mg/kg) administration (SC injection). Induced animals demonstrated severely elevated mean PA pressures and developed neointima and smooth muscle hypertrophy. At week 4, PAH induced rats were treated with Anc80 AC (1×10¹¹ genome copies). At week 6 and 8, animals were validated by MRI for heart function and RV and PA catheterization for pressure measurement. After AC administration right ventricular systolic volume decreased 39%.

FIG. 9 shows hemodynamic data after Acn80 AC intra-tracheal injection. Rats were subjected to PAH induction protocol. On week 0 Rat were subjected to baseline MRI, RV and PA catheterization to measure the pressure, and Left lung removal. On day 7 pneumonectomized rats were subjected to SU5416 (Su/Pn 10 mg/kg) administration (SC injection). Induced animals demonstrated severely elevated mean PA pressures and develop neointima and smooth muscle hypertrophy. On week 4 PAH induced rats were treated with Anc80 AC (1×10¹¹genome copies). On week 6 and 8 animal were validated by MRI for heart function and RV and PA catheterization for pressure measurement. After AC administration right ventricular ejection fraction increased in 65%.

FIG. 10 shows hemodynamic data after Anc80 AC intra-tracheal injection. Rat were subjected to PAH induced protocol. On week 0 Rat were subjected to baseline MRI, RV and PA catheterization to measure the pressure, and Left lung removal. On day 7 pneumonectomized rats were subjected to SU5416 (Su/Pn 10 mg/kg) administration (SC injection). Induced animals demonstrated severely elevated mean PA pressures and develop neointima and smooth muscle hypertrophy. On week 4 PAH induced rats were treated with Anc80 AC (1×10¹¹ genome copies). On week 6 and 8 animal were validated by MRI for heart function and RV and PA catheterization for pressure measurement. After AC administration mean pulmonary artery pressure decreased 94%.

FIG. 11 shows hemodynamic data after Anc80 AC intra-tracheal injection. Rat were subjected to PAH induce protocol. On week 0 Rat were subjected to baseline MRI, RV and PA catheterization to measure the pressure, and Left lung removal. On day 7 pneumonectomized rats were subjected to SU5416 (Su/Pn 10 mg/kg) administration (SC injection). Induced animals demonstrated severely elevated mean PA pressures and develop neointima and smooth muscle hypertrophy. On week 4 PAH induced rats were treated with Anc80 AC (1×10¹¹ genome copies). On week 6 and 8 animal were validated by MRI for heart function and RV and PA catheterization for pressure measurement. After AC administration mean pulmonary vascular resistance decreased 4.8 times.

FIG. 12 shows MRI images showing heart function after Anc80 AC intra-tracheal injection. Rats were subjected to PAH induction protocol. On week 0, rats were subjected to baseline MRI, RV and PA catheterization to measure the pressure, and left lung removal. On day 7, pneumonectomized rats were subjected to SU5416 (Su/Pn 10 mg/kg) administration (SC injection). Induced animals demonstrated severely elevated mean PA pressures and developed neointima and smooth muscle hypertrophy. On week 4, PAH-induced rats were treated with Anc80 AC (1×10¹¹ genome copies). On week 6 and 8, animals were evaluated by MRI for heart function and RV and PA catheterization for pressure measurement. Animals treated with Anc80 AC at 8 weeks showed excellent cardiac function.

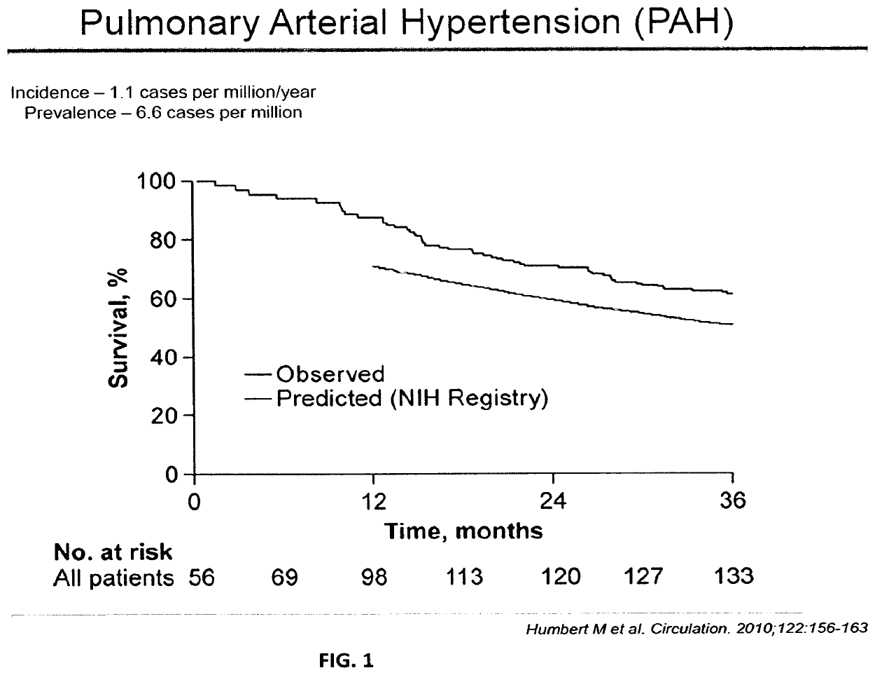

### DETAILED DESCRIPTION OF THE DISCLOSURE

All patents, published applications and other references cited herein are hereby incorporated by reference into the present application.

In the description that follows, certain conventions will be followed as regards the usage of terminology. In general, terms used herein are intended to be interpreted consistently with the meaning of those terms, as they are known to those of skill in the art. Some definitions are provided purely for the convenience of the reader.

The term “cell or group of cells” is intended to encompass single cells as well as multiple cells either in suspension or in monolayers. Whole tissues also constitute a group of cells.

The term “ischemic” as it is known in the art refers to a deficiency in the supply of blood to a part of the body (such as the heart, brain or other organ/tissue) that is due to obstruction of the inflow of arterial blood as by the narrowing of arteries by spasm or disease.

The term “inhibit” or “inhibition” when used in conjunction with a discussion of senescence includes the ability of the sphingolipid-metabolizing proteins of the disclosure to reverse senescence, thereby returning to normal or near normal function.

The terms “stress”, “stress-related events” or “cellular-stress” refers to a wide range of molecular changes that cells undergo in response to environmental stressors, such as extreme temperatures, exposure to toxins, mechanical damage, anoxia, and noise.

### Pulmonary Arterial Hypertension

Pulmonary arterial hypertension (PAH) is one form of a broader condition known as pulmonary hypertension, which means high blood pressure in the lungs. In PAH, the rise in blood pressure is caused by changes in the cells that line the pulmonary arteries. These changes can cause the walls of the arteries to become stiff and thick, and extra tissue may form. The blood vessels may also become inflamed and tight. In many cases of pulmonary arterial hypertension, the cause is idiopathic (i.e., unknown). Other causes include heart abnormalities present at birth, HIV infection (Group I PAH); left-sided valvular heart disease such as mitral valve or aortic valve disease (Group 2 PAH); chronic obstructive pulmonary disease and other lung disease (Group 3 PAH); connective tissue/autoimmune disorders (such as scleroderma) and others.

PAH occurs when the very small arteries throughout the lungs narrow in diameter, which increases the resistance to blood flow through the lungs. Over time, the increased blood pressure can damage the heart. A number of diseases and conditions can cause PAH, and symptoms are similar to the symptoms often seen in more common diseases, such as asthma, chronic obstructive pulmonary disease (COPD), and heart failure.

### Mitral Valve Prolapse

Mitral Valve Prolapse (MVP) is a common disorder afflicting at least 2% to 3% of the general population that affects ≈7.8 million individuals in the United States and >176 million people worldwide [Freed L A 1999, Devereux R B, 2001].

A canine model of a related disease, Myxomatous Mitral Valve Degeneration, MMVD, is used to further understanding of the role of Anc80 delivery of sphingolipid—metabolizing proteins in MVP.

The present technology is based on the use of sphingolipid metabolizing proteins in order to manipulate the fate of cells post stress-related events and during disease and aging. Different types of stress can initiate the signal transduction that leads to two major pathways: one can lead to cell death and the other leads to senescence, which is characterized by low cell function and arrested regeneration and amplification. In addition, senescent cells secrete different factors that can trigger an immune response and lead to inflammation and additional cell death. Cell senescence can be initiated not only by stress but also during aging. Both the cell death and cell senescence pathways involve sphingolipid metabolism mainly an increase in ceramide that can lead to both.

Ceramide has been shown to induce apoptotic cell death in different cells type including murine and human cardiomyocytes. On the other hand, sphingosine, one of the products of ceramide degradation can be phosphorylated to give rise to a major agent of cell survival and cardioprotection, sphingosine 1 phosphate.

There are also several studies that support association of the signaling lipid, ceramide, and its metabolizing enzymes with cellular and organismal aging and senescence. It has been reported that the intracellular level of ceramide increased during stress related signaling such as cell culture and aging.

Ceramidase, for example, acid ceramidase (AC) is required to hydrolyze ceramide into sphingosine and free fatty acids. Sphingosine is rapidly converted to sphingosine-1-phosphate (S1P), another important signaling lipid that counteracts the effects of ceramide and promotes cell survival. Thus, AC acts as a “rheostat” that regulates the levels of ceramide and S1P in cells, and as such participates in the complex and delicate balance between death and survival.

We have previously shown that AC expression is carefully regulated during oocyte maturation and early embryo development (Eliyahu, et al, 2010). We have also found that the complete “knock-out” of AC function in mice leads to embryo death between the 2 and 8-cell stage (Eliyahu, FASEB J, 2007). In addition, our previous publication (Eliyahu, FASEB J, 2010) showed that the ceramide-metabolizing enzyme, AC is expressed and active in human cumulus cells and follicular fluid, essential components of this environment, and that the levels of this enzyme are positively correlated with the quality of human embryos formed in vitro. These observations led to a new approach for oocyte and embryo culture that markedly improves the outcome of in vitro fertilization (IVF).

In this disclosure, we describe a strategy to reduce pulmonary arterial hypertension by increasing ceramide hydrolysis by overexpression of acid ceramidase. With this strategy, not only can we reduce ceramide levels but we also increase the reservoir of sphingosine which is the main building block for the pro-survival molecule sphingosine-1-phosphate (S1P).

### Choice of Vehicle and Duration of Expression Needed

Methods and compositions for in vivo delivery of a construct that expresses a sphingolipid-metabolizing protein such as ceramidase were explored. For applications where more sustained expression of a sphingolipid metabolizing enzyme is required, expression from an Anc80 vector may be desirable.

Adeno-associated viruses have emerged as one of the most promising vectors in the field of gene therapy. Preclinical and clinical studies have validated the use of adeno-associated viral vectors (AAVs) as a safe and efficient delivery vehicle for gene transfer. AAV vectors are known to be expressed for several months or longer post administration; thus, they provide a more extensive time frame than modRNA.

More recently, Zinn et al. identified Anc80 as a highly potent in vivo gene therapy vector for targeting liver, muscle and retina. Anc80 virus, an in silico designed gene therapy vector, has demonstrated high gene expression levels in the liver, eye and ear compared to naturally occurring adeno-associated viral vectors (AAVs) that are currently in clinical development. Due to its synthetic nature, Anc80 does not circulate in humans, making it less likely to be recognized immunologically by antibodies against naturally-occurring AAVs. Anc80 also provides longer lasting expression. In addition, Anc80 expresses protein in much higher amounts than AAVs, so the amount of necessary virus is much less that leads to lower immune response.

The present disclosure, therefore, also provides a method for inhibiting or reducing pulmonary arterial hypertension by administration of a cocktail of Anc80 virus encoding sphingolipid metabolizing proteins. The treatment includes different combinations of Acid Ceramidase (AC) and/or Sphingosine Kinase (SPHK) and/or Sphingosine-1-phosphate receptor (S1PR) gene (cDNA). Anc80 virus, an in silico designed gene therapy vector, Anc80 has demonstrated high gene expression levels in the liver, eye and ear compared to naturally-occurring adeno-associated viral vectors (AAVs) that are currently in clinical development. Anc80, an engineered gene therapy vector, is synthetic in nature and has been shown to reduce cross-reactivity with commonly used AAV vectors. Anc80 is a potent gene therapy vector that is not known to circulate in humans, making it less likely to cross-react immunologically with naturally occurring AAVs.

### Sphinqolipid-Metabolizing Proteins

In one embodiment, a composition useful for practicing the method of the present disclosure may include either individually or in different combinations Anc80 vectors encoding the following sphingolipid-metabolizing proteins: ceramidase (acid, neutral or alkaline), sphingosine kinase (SPHK), sphingosine-1-phosphate receptor (S1PR), and a ceramide kinase (CERK). In one embodiment, the sphingolipid-metabolizing protein is a ceramidase.

Ceramidase is an enzyme that cleaves fatty acids from ceramide, producing sphingosine (SPH), which in turn is phosphorylated by a sphingosine kinase to form sphingosine-1-phosphate (S1P). Ceramidase is the only enzyme that can regulate ceramide hydrolysis to prevent cell death and SHPK is the only enzyme that can synthesize sphingosine 1 phosphate (S1P) from sphingosine (the ceramide hydrolysis product) to initiate cell survival. S1 PR, a G protein-coupled receptor binds the lipid-signaling molecule S1P to induce cell proliferation, survival, and transcriptional activation. CERK is an phosphatase that phosphorylates ceramide into ceramide 1 phosphate to induce cell survival.

Presently, 7 human ceramidases encoded by 7 distinct genes have been cloned:
acid ceramidase (ASAH1)—associated with cell survival;
neutral ceramidase (ASAH2, ASAH2B, ASAH2C)—protective against inflammatory cytokines;
alkaline ceramidase 1 (ACER1)—mediating cell differentiation by controlling the generation of SPH and S1P;
alkaline ceramidase 2 (ACER2)—important for cell proliferation and survival; and
alkaline ceramidase 3 (ACER3).

The nucleotide sequences for nucleic acids encoding these ceramidases are shown in Table 1.

In one embodiment, Anc80, a relatively nascent technology, has shown considerable potential as a delivery vehicle for gene therapy in disease, for example, cardiac disease, hearing loss, vision loss and neurodegenerative diseases. Anc80 as an engineered gene therapy vector is synthetic in nature and is not known to circulate in humans. It has been shown to have reduced cross-reactivity with commonly used AAV vectors. Anc80 therefore is a potent gene therapy vector, which is less likely to be recognized immunologically by antibodies against naturally occurring AAVs.

### Advantages

An Anc80 vector encoding acid ceramidase (Anc80.AC) has multiple advantages over other potential anti-apoptotic factors.

### Low toxicity

Low or no toxicity: The AC protein, by itself, is not toxic. Physiological enzymes are not expected to have toxic effects. The biological function of AC is the control of ceramide metabolism has no direct influence other cellular signaling. Treated cells present only a modest increase in AC generation in cells post gene therapy treatment. The AC protein level expressed after treatment is far below extraordinarily high levels reported in aberrant diseased cells with poorly understood mechanisms. The AC protein exists in two forms, and undergoes a transformation from an inactive to active form in the cell. The inactive AC precursor undergoes an auto-self cleavage to the active enzyme, which is responsible for hydrolyzing ceramide to sphingosine. This exquisitely evolved self-regulating mechanism, call the Sphingolipid Rheostat, regulates, by hydrolysis toxic levels of ceramides in the cell after exposure to stress. The transfection of cells with Anc80.AC can increase the cellular reservoir of inactive precursor, thereby allowing physiological sphingolipid levels to regulate the conversion to the active AC enzyme necessary for cellular robustness and organism survival. In addition, Eliyahu lab created mouse model that is constantly overexpressing the AC enzyme (COEAC) in all tissues. The COEAC mice viability provides evidence that AC is a non-toxic protein.

### Ease of delivery

As mentioned, Anc80, an engineered gene therapy vector, is synthetic in nature and shown to reduce cross-reactivity with commonly used AAV vectors. Anc80 is a potent gene therapy vector that is not known to circulate in humans, making it less likely to be recognized immunologically by antibodies against naturally occurring AAVs. Recently, it has been shown successful, robust, transfection of Anc80 virus into liver, eye and ear tissue in vivo (see Magali Trayssac, Yusuf A. Hannun, and Lina M. Obeid. Role of sphingolipids in senescence: implication in aging and age-related diseases. J. Clin. Inves. 2018;128(7):2702-2712., which is incorporated herein by reference.)

In one embodiment, Anc80.AC is administered to at-risk tissue by aerosolization of a composition comprising an Anc80 viral vector that codes for the expression of acid ceram idase.

### Unique Physiological Function of Acid Ceramidase

Increase in ceramide level can have different outcomes leading to cell death and/or senescence. Ceramidase is the only enzyme that can hydrolyze ceramide and therefore, the only enzyme that can directly decrease the levels of ceramide in cells.

Table 1 contains the nucleotide sequences to be encoded by the vectors disclosed for use in practicing the method.

TABLE 1

| Gene | Open Reading Frame |
|---|---|
| ASAH1 | ATGCCGGGCCGGAGTTGCGTCGCCTTAGTC |
| transcript | CTCCTGGCTGCCGCCGTCAGCTGTGCCGTC |
| variant 1 | GCGCAGCACGCGCCGCCGTGGACAGAGGAC |
| (ACv1) | TGCAGAAAATCAACCTATCCTCCTTCAGGA |
| | CCAACGTACAGAGGTGCAGTTCCATGGTAC |
| | ACCATAAATCTTGACTTACCACCCTACAAA |
| | AGATGGCATGAATTGATGCTTGACAAGGCA |
| | CCAGTGCTAAAGGTTATAGTGAATTCTCTG |
| | AAGAATATGATAAATACATTCGTGCCAAGT |
| | GGAAAAATTATGCAGGTGGTGGATGAAAAA |
| | TTGCCTGGCCTACTTGGCAACTTTCCTGGC |
| | CCTTTGAAGAGGAAATGAAGGGTATTGCCG |
| | CTGTTACTGATATACCTTTAGGAGAGATTA |
| | TTTCATTCAATATTTTTTATGAATTATTTA |
| | CCATTTGTACTTCAATAGTAGCAGAAGACA |
| | AAAAAGGTCATCTAATACATGGGAGAAACA |
| | TGGATTTTGGAGTATTTCTTGGGTGGAACA |
| | TAAATAATGATACCTGGGTCATAACTGAGC |
| | AACTAAAACCTTTAACAGTGAATTTGGATT |
| | TCCAAAGAAACAACAAAACTGTCTTCAAGG |
| | CTTCAAGCTTTGCTGGCTATGTGGGCATGT |
| | TAACAGGATTCAAACCAGGACTGTTCAGTC |
| | TTACACTGAATGAACGTTTCAGTATAAATG |
| | GTGGTTATCTGGGTATTCTAGAATGGATTC |
| | TGGGAAAGAAAGATGTCATGTGGATAGGGT |
| | TCCTCACTAGAACAGTTCTGGAAAATAGCA |
| | CAAGTTATGAAGAAGCCAAGAATTTATTGA |
| | CCAAGACCAAGATATTGGCCCCAGCCTACT |
| | TTATCCTGGGAGGCAACCAGTCTGGGGAAG |
| | GTTGTGTGATTACACGAGACAGAAAGGAAT |
| | CATTGGATGTATATGAACTCGATGCTAAGC |
| | AGGGTAGATGGTATGTGGTACAAACAAATT |
| | ATGACCGTTGGAAACATCCCTTCTTCCTTG |
| | ATGATCGCAGAACGCCTGCAAAGATGTGTC |
| | TGAACCGCACCAGCCAAGAGAATATCTCAT |
| | TTGAAACCATGTATGATGTCCTGTCAACAA |
| | AACCTGTCCTCAACAAGCTGACCGTATACA |
| | CAACCTTGATAGATGTTACCAAAGGTCAAT |
| | TCGAAACTTACCTGCGGGACTGCCCTGACC |
| | CTTGTATAGGTTGGTGA |
| | (SEQ ID NO: 1) |
| Sphk1 | ATGGATCCAGTGGTCGGTTGCGGACGTGGC |
| | CTCTTTGGTTTTGTTTTCTCAGCGGGCGGC |
| | CCCCGGGGCGTGCTCCCGCGGCCCTGCCGC |
| | GTGCTGGTGCTGCTGAACCCGCGCGGCGGC |
| | AAGGGCAAGGCCTTGCAGCTCTTCCGGAGT |
| | CACGTGCAGCCCCTTTTGGCTGAGGCTGAA |
| | ATCTCCTTCACGCTGATGCTCACTGAGCGG |
| | CGGAACCACGCGCGGGAGCTGGTGCGGTCG |
| | GAGGAGCTGGGCCGCTGGGACGCTCTGGTG |
| | GTCATGTCTGGAGACGGGCTGATGCACGAG |
| | GTGGTGAACGGGCTCATGGAGCGGCCTGAC |
| | TGGGAGACCGCCATCCAGAAGCCCCTGTGT |
| | AGCCTCCCAGCAGGCTCTGGCAACGCGCTG |
| | GCAGCTTCCTTGAACCATTATGCTGGCTAT |
| | GAGCAGGTCACCAATGAAGACCTCCTGACC |
| | AACTGCACGCTATTGCTGTGCCGCCGGCTG |
| | CTGTCACCCATGAACCTGCTGTCTCTGCAC |
| | ACGGCTTCGGGGCTGCGCCTCTTCTCTGTG |
| | CTCAGCCTGGCCTGGGGCTTCATTGCTGAT |
| | GTGGACCTAGAGAGTGAGAAGTATCGGCGT |
| | CTGGGGGAGATGCGCTTCACTCTGGGCACC |
| | TTCCTGCGTCTGGCAGCCCTGCGCACCTAC |
| | CGCGGCCGACTGGCCTACCTCCCTGTAGGA |
| | AGAGTGGGTTCCAAGACACCTGCCTCCCCC |
| | GTTGTGGTCCAGCAGGGCCCGGTAGATGCA |
| | CACCTTGTGCCACTGGAGGAGCCAGTGCCC |
| | TCTCACTGGACAGTGGTGCCCGACGAGGAC |
| | TTTGTGCTAGTCCTGGCACTGCTGCACTCG |
| | CACCTGGGCAGTGAGATGTTTGCTGCACCC |
| | ATGGGCCGCTGTGCAGCTGGCGTCATGCAT |
| | CTGTTCTACGTGCGGGCGGGAGTGTCTCGT |
| | GCCATGCTGCTGCGCCTCTTCCTGGCCATG |
| | GAGAAGGGCAGGCATATGGAGTATGAATGC |
| | CCCTACTTGGTATATGTGCCCGTGGTCGCC |
| | TTCCGCTTGGAGCCCAAGGATGGGAAAGGT |
| | GTGTTTGCAGTGGATGGGGAATTGATGGTT |
| | AGCGAGGCCGTGCAGGGCCAGGTGCACCCA |
| | AACTACTTCTGGATGGTCAGCGGTTGCGTG |
| | GAGCCCCCGCCCAGCTGGAAGCCCCAGCAG |
| | ATGCCACCGCCAGAAGAGCCCTTATGA |
| | (SEQ ID NO: 2) |
| S1PR2 | ATGGGCAGCTTGTACTCGGAGTACCTGAAC |
| | CCCAACAAGGTCCAGGAACACTATAATTAT |
| | ACCAAGGAGACGCTGGAAACGCAGGAGACG |
| | ACCTCCCGCCAGGTGGCCTCGGCCTTCATC |
| | GTCATCCTCTGTTGCGCCATTGTGGTGGAA |
| | AACCTTCTGGTGCTCATTGCGGTGGCCCGA |
| | AACAGCAAGTTCCACTCGGCAATGTACCTG |
| | TTTCTGGGCAACCTGGCCGCCTCCGATCTA |
| | CTGGCAGGCGTGGCCTTCGTAGCCAATACC |
| | TTGCTCTGGCTCTGTCACGCTGAGGCTGAC |
| | GCCTGTGCAGTGGTTTGCCCGGGAGGGCTC |
| | TGCCTTCATCACGCTCTCGGCCTCTGTCTT |
| | CAGCCTCCTGGCCATCGCCATTGAGCGCCA |
| | CGTGGCCATTGCCAAGGTCAAGCTGTATGG |
| | CAGCGACAAGAGCTGCCGCATGCTTCTGCT |
| | CATCGGGGCCTCGTGGCTCATCTCGCTGGT |
| | CCTCGGTGGCCTGCCCATCCTTGGCTGGAA |
| | CTGCCTGGGCCACCTCGAGGCCTGCTCCAC |
| | TGTCCTGCCTCTCTACGCCAAGCATTATGT |
| | GCTGTGCGTGGTGACCATCTTCTCCATCAT |
| | CCTGTTGGCCATCGTGGCCCTGTACGTGCG |
| | CATCTACTGCGTGGTCCGCTCAAGCCACGC |
| | TGACATGGCCGCCCCGCAGACGCTAGCCCT |
| | GCTCAAGACGGTCACCATCGTGCTAGGCGT |
| | CTTTATCGTCTGCTGGCTGCCCGCCTTCAG |
| | CATCCTCCTTCTGGACTATGCCTGTCCCGT |
| | CCACTCCTGCCCGATCCTCTACAAAGCCCA |
| | CTACTTTTTCGCCGTCTCCACCCTGAATTC |
| | CCTGCTCAACCCCGTCATCTACACGTGGCG |
| | CAGCCGGGACCTGCGGCGGGAGGTGCTTCG |
| | GCCGCTGCAGTGCTGGAGGCCGGGGGTGGG |
| | GGTGCAAGGACGGAGGCGGGGCGGGACCCC |
| | GGGCCACCACCTCCTGCCACTCCGCAGCTC |
| | CAGCTCCCTGGAGAGGGGCATGCACATGCC |
| | CACGTCACCCACGTTTCTGGAGGGCAACAC |
| | GGTGGTCATG |
| | (SEQ ID NO: 3) |
| Firefly | ATGGCCGATGCTAAGAACATTAAGAAGGGC |
| luciferase | CCTGCTCCCTTCTACCCTCTGGAGGATGGC |
| | ACCGCTGGCGAGCAGCTGCACAAGGCCATG |
| | AAGAGGTATGCCCTGGTGCCTGGCACCATT |
| | GCCTTCACCGATGCCCACATTGAGGTGGAC |
| | ATCACCTATGCCGAGTACTTCGAGATGTCT |
| | GTGCGCCTGGCCGAGGCCATGAAGAGGTAC |
| | GGCCTGAACACCAACCACCGCATCGTGGTG |
| | TGCTCTGAGAACTCTCTGCAGTTCTTCATG |
| | CCAGTGCTGGGCGCCCTGTTCATCGGAGTG |
| | GCCGTGGCCCCTGCTAACGACATTTACAAC |
| | GAGCGCGAGCTGCTGAACAGCATGGGCATT |
| | TCTCAGCCTACCGTGGTGTTCGTGTCTAAG |
| | AAGGGCCTGCAGAAGATCCTGAACGTGCAG |
| | AAGAAGCTGCCTATCATCCAGAAGATCATC |
| | ATCATGGACTCTAAGACCGACTACCAGGGC |
| | TTCCAGAGCATGTACACATTCGTGACATCT |
| | CATCTGCCTCCTGGCTTCAACGAGTACGAC |
| | TTCGTGCCAGAGTCTTTCGACAGGGACAAA |
| | ACCATTGCCCTGATCATGAACAGCTCTGGG |
| | TCTACCGGCCTGCCTAAGGGCGTGGCCCTG |
| | CCTCATCGCACCGCCTGTGTGCGCTTCTCT |
| | CACGCCCGCGACCCTATTTTCGGCAACCAG |
| | ATCATCCCCGACACCGCTATTCTGAGCGTG |
| | GTGCCATTCCACCACGGCTTCGGCATGTTC |
| | ACCACCCTGGGCTACCTGATTTGCGGCTTT |
| | CGGGTGGTGCTGATGTACCGCTTCGAGGAG |
| | GAGCTGTTCCTGCGCAGCCTGCAAGACTAC |
| | AAAATTCAGTCTGCCCTGCTGGTGCCAACC |
| | CTGTTCAGCTTCTTCGCTAAGAGCACCCTG |
| | ATCGACAAGTACGACCTGTCTAACCTGCAC |
| | GAGATTGCCTCTGGCGGCGCCCCACTGTCT |
| | AAGGAGGTGGGCGAAGCCGTGGCCAAGCGC |
| | TTTCATCTGCCAGGCATCCGCCAGGGCTAC |
| | GGCCTGACCGAGACAACCAGCGCCATTCTG |
| | ATTACCCCAGAGGGCGACGACAAGCCTGGC |
| | GCCGTGGGCAAGGTGGTGCCATT |
| | CTTCGAGGCCAAGGTGGTGGACCTGGACAC |
| | CGGCAAGACCCTGGGAGTGAACCAGCGCGG |
| | CGAGCTGTGTGTGCGCGGCCCTATGATTAT |
| | GTCCGGCTACGTGAATAACCCTGAGGCCAC |
| | AAACGCCCTGATCGACAAGGACGGCTGGCT |
| | GCACTCTGGCGACATTGCCTACTGGGACGA |
| | GGACGAGCACTTCTTCATCGTGGACCGCCT |
| | GAAGTCTCTGATCAAGTACAAGGGCTACCA |
| | GGTGGCCCCAGCCGAGCTGGAGTCTATCCT |
| | GCTGCAGCACCCTAACATTTTCGACGCCGG |
| | AGTGGCCGGCCTGCCCGACGACGATGCCGG |
| | CGAGCTGCCTGCCGCCGTCGTCGTGCTGGA |
| | ACACGGCAAGACCATGACCGAGAAGGAGAT |
| | CGTGGACTATGTGGCCAGCCAGGTGACAAC |
| | CGCCAAGAAGCTGCGCGGCGGAGTGGTGTT |
| | CGTGGACGAGGTGCCCAAGGGCCTGACCGG |
| | CAAGCTGGACGCCCGCAAGATCCGCGAGAT |
| | CCTGATCAAGGCTAAGAAAGGCGGCAAGAT |
| | CGCCGTGTAA |
| | (SEQ ID NO: 4) |
| nGFP | ATGGTGAGCAAGGGCGAGGAGCTGTTCACC |
| | GGGGTGGTGCCCATCCTGGTCGAGCTGGAC |
| | GGCGACGTAAACGGCCACAAGTTCAGCGTG |
| | TCCGGCGAGGGCGAGGGCGATGCCACCTAC |
| | GGCAAGCTGACCCTGAAGTTCATCTGCACC |
| | ACCGGCAAGCTGCCCGTGCCCGTGGCCCAC |
| | CCTCGTGACCACCCTGACCTACGGCGTGCA |
| | GTGCTTCAGCCGCTACCCCGACCACATGAA |
| | GCAGCACGACTTCTTCAAGTCCGCCATGCC |
| | CGAAGGCTACGTCCAGGAGCGCACCATCTT |
| | CTTCAAGGACGACGGCAACTACAAGACCCG |
| | CGCCGAGGTGAAGTTCGAGGGCGACACCCT |
| | GGTGAACCGCATCGAGCTGAAGGGCATCGA |
| | CTTCAAGGAGGACGGCAACATCCTGGGGCA |
| | CAAGCTGGAGTACAACTACAACAGCCACAA |
| | CGTCTATATCATGGCCGACAAGCAGAAGAA |
| | CGGCATCAAGGTGAACTTCAAGATCCGCCA |
| | CAACATCGAGGACGGCAGCGTGCAGCTCGC |
| | CGACCACTACCAGCAGAACACCCCCATCGG |
| | CGACGGCCCCGTGCTGCTGCCCGACAACCA |
| | CTACCTGAGCACCCAGTCCGCCCTGAGCAA |
| | AGACCCCAACGAGAAGCGCGATCACATGGT |
| | CCTGCTGGAGTTCGTGACCGCCGCCGGGAT |
| | CACTCTCGGCATGGACGAGCTGTACAAGGG |
| | AGATCCAAAAAAGAAGAGAAAGGTAGGCGA |
| | TCCAAAAAAGAAGAGAAAGGTAGGTGATCC |
| | AAAAAAGAAGAGAAAGGTATAA |
| | (SEQ ID NO: 5) |
| ASAH2 | ATGAACTGCTGCATCGGGCTGGGAGAGAAA |
| transcript | GCTCGCGGGTCCCACCGGGCCTCCTACCCA |
| variant 2 | AGTCTCAGCGCGCTTTTCACCGAGGCCTCA |
| (ACv2) | ATTCTGGGATTTGGCAGCTTTGCTGTGAAA |
| | GCCCAATGGACAGAGGACTGCAGAAAATCA |
| | ACCTATCCTCCTTCAGGACCAACGTACAGA |
| | GGTGCAGTTCCATGGTACACCATAAATCTT |
| | GACTTACCACCCTACAAAAGATGGCATGAA |
| | TTGATGCTTGACAAGGCACCAGTGCTAAAG |
| | GTTATAGTGAATTCTCTGAAGAATATGATA |
| | AATACATTCGTGCCAAGTGGAAAAATTATG |
| | CAGGTGGTGGATGAAAAATTGCCTGGCCTA |
| | CTTGGCAACTTTCCTGGCCCTTTTGAAGAG |
| | GAAATGAAGGGTATTGCCGCTGTTACTGAT |
| | ATACCTTTAGGAGAGATTATTTCATTCAAT |
| | ATTTTTTATGAATTATTTACCATTTGTACT |
| | TCAATAGTAGCAGAAGACAAAAAAGGTCAT |
| | CTAATACATGGGAGAAACATGGATTTTGGA |
| | GTATTTCTTGGGTGGAACATAAATAATGAT |
| | ACCTGGGTCATAACTGAGCAACTAAAACCT |
| | TTAACAGTGAATTTGGATTTCCAAAGAAAC |
| | AACAAAACTGTCTTCAAGGCTTCAAGCTTT |
| | GCTGGCTATGTGGGCATGTTAACAGGATTC |
| | AAACCAGGACTGTTCAGTCTTACACTGAAT |
| | GAACGTTTCAGTATAAATGGTGGTTATCTG |
| | GGTATTCTAGAATGGATTCTGGGAAAGAAA |
| | GATGTCATGTGGATAGGGTTCCTCACTAGA |
| | ACAGTTCTGGAAAATAGCACAAGTTATGAA |
| | GAAGCCAAGAATTTATTGACCAAGACCAAG |
| | ATATTGGCCCCAGCCTACTTTATCCTGGGA |
| | GGCAACCAGTCTGGGGAAGGTTGTGTGATT |
| | ACACGAGACAGAAAGGAATCATTGGATGTA |
| | TATGAACTCGATGCTAAGCAGGGTAGATGG |
| | TATGTGGTACAAACAAATTATGACCGTTGG |
| | AAACATCCCTTCTTCCTTGATGATCGCAGA |
| | ACGCCTGCAAAGATGTGTCTGAACCGCACC |
| | AGCCAAGAGAATATCTCATTTGAAACCATG |
| | TATGATGTCCTGTCAACAAAACCTGTCCTC |
| | AACAAGCTGACCGTATACACAACCTTGATA |
| | GATGTTACCAAAGGTCAATTCGAAACTTAC |
| | CTGCGGGACTGCCCTGACCCTTGTATAGGT |
| | TGGTGA |
| | (SEQ ID NO: 6) |
| ASAH1 | ATGAACTGCTGCATCGGGCTGGGAGAGAAA |
| transcript | GCTCGCGGGTCCCACCGGGCCTCCTACCCA |
| variant 3 | AGTCTCAGCGCGCTTTTCACCGAGGCCTCA |
| | ATTCTGGGATTTGGCAGCTTTGCTGTGAAA |
| | GCCCAATGGACAGAGGACTGCAGAAAATCA |
| | ACCTATCCTCCTTCAGGACCAACTGTCTTC |
| | CCTGCTGTTATAAGGTACAGAGGTGCAGTT |
| | CCATGGTACACCATAAATCTTGACTTACCA |
| | CCCTACAAAAGATGGCATGAATTGATGCTT |
| | GACAAGGCACCAGTGCCTGGCCTACTTGGC |
| | AACTTTCCTGGCCCTTTTGAAGAGGAAATG |
| | AAGGGTATTGCCGCTGTTACTGATATACCT |
| | TTAGGAGAGATTATTTCATTCAATATTTTT |
| | TATGAATTATTTACCATTTGTACTTCAATA |
| | GTAGCAGAAGACAAAAAAGGTCATCTAATA |
| | CATGGGAGAAACATGGATTTTGGAGTATTT |
| | TCTTGGGTGGAACATAAATAATGATACCTG |
| | GGTCATAACTGAGCAACTAAAACCTTTAAC |
| | AGTGAATTTGGATTTCCAAAGAAACAACAA |
| | AACTGTCTTCAAGGCTTCAAGCTTTGCTGG |
| | CTATGTGGGCATGTTAACAGGATTCAAACC |
| | AGGACTGTTCAGTCTTACACTGAATGAACG |
| | TTTCAGTATAAATGGTGGTTATCTGGGTAT |
| | TCTAGAATGGATTCTGGGAAAGAAAGATGT |
| | CATGTGGATAGGGTTCCTCACTAGAACAGT |
| | TCTGGAAAATAGCACAAGTTATGAAGAAGC |
| | CAAGAATTTATTGACCAAGACCAAGATATT |
| | GGCCCCAGCCTACTTTATCCTGGGAGGCAA |
| | CCAGTCTGGGGAAGGTTGTGTGATTACACG |
| | AGACAGAAAGGAATCATTGGATGTATATGA |
| | ACTCGATGCTAAGCAGGGTAGATGGTATGT |
| | GGTACAAACAAATTATGACCGTTGGAAACA |
| | TCCCTTCTTCCTTGATGATCGCAGAACGCC |
| | TGCAAAGATGTGTCTGAACCGCACCAGCCA |
| | AGAGAATATCTCATTTGAAACCATGTATGA |
| | TGTCCTGTCAACAAAACCTGTCCTCAACAA |
| | GCTGACCGTATACACAACCTTGATAGATGT |
| | TACCAAAGGTCAATTCGAAACTTACCTGCG |
| | GGACTGCCCTGACCCTTGTATAGGTTGGTG |
| | A |
| | (SEQ ID NO: 7) |
| ASAH2 | ATGGCCAAACGCACCTTCTCTAACTTGGAG |
| transcript | ACATTCCTGATTTTCCTCCTTGTAATGATG |
| variant 1 | AGTGCCATCACAGTGGCCCTTCTCAGCCTC |
| | TTGTTTATCACCAGTGGGACCATTGAAAAC |
| | CACAAAGATTTAGGAGGCCATTTTTTTTCA |
| | ACCACCCAAAGCCCTCCAGCCACCCAGGGC |
| | TCCACAGCTGCCCAACGCTCCACAGCCACC |
| | CAGCATTCCACAGCCACCCAGAGCTCCACA |
| | GCCACTCAAACTTCTCCAGTGCCTTTAACC |
| | CCAGAGTCTCCTCTATTTCAGAACTTCAGT |
| | GGCTACCATATTGGTGTTGGACGAGCTGAC |
| | TGCACAGGACAAGTAGCAGATATCAATTTG |
| | ATGGGCTATGGCAAATCCGGCCAGAATGCA |
| | CAGGGCATCCTCACCAGGCTATACAGTCGT |
| | GCCTTCATCATGGCAGAACCTGATGGGTCC |
| | AATCGAACAGTGTTTGTCAGCATCGACATA |
| | GGCATGGTATCACAAAGGCTCAGGCTGGAG |
| | GTCCTGAACAGACTGCAGAGTAAATATGGC |
| | TCCCTGTACAGAAGAGATAATGTCATCCTG |
| | AGTGGCACTCACACTCATTCAGGTCCTGCA |
| | GGATATTTCCAGTATACCGTGTTTGTAATT |
| | GCCAGTGAAGGATTTAGCAATCAAACTTTT |
| | CAGCACATGGTCACTGGTATCTTGAAGAGC |
| | ATTGACATAGCACACACAAATATGAAACCA |
| | GGCAAAATCTTCATCAATAAAGGAAATGTG |
| | GATGGTGTGCAGATCAACAGAAGTCCGTAT |
| | TCTTACCTTCAAAATCCGCAGTCAGAGAGA |
| | GCAAGGTATTCTTCAAATACAGACAAGGAA |
| | ATGATAGTTTTGAAAATGGTAGATTTGAAT |
| | GGAGATGACTTGGGCCTTATCAGCTGGTTT |
| | GCCATCCACCCGGTCAGCATGAACAACAGT |
| | AACCATCTTGTAAACAGTGACAATGTGGGC |
| | TATGCATCTTACCTGCTTGAGCAAGAGAAG |
| | AACAAAGGATATCTACCTGGACAGGGGCCA |
| | TTTGTAGCAGCCTTTGCTTCATCAAACCTA |
| | GGAGATGTGTCCCCCAACATTCTTGGACCA |
| | CGTTGCATCAACACAGGAGAGTCCTGTGAT |
| | AACGCCAATAGCACTTGTCCCATTGGTGGG |
| | CCTAGCATGTGCATTGCTAAGGGACCTGGA |
| | CAGGATATGTTTGACAGCACACAAATTATA |
| | GGACGGGCCATGTATCAGAGAGCAAAGGAA |
| | CTCTATGCCTCTGCCTCCCAGGAGGTAACA |
| | GGACCACTGGCTTCAGCACACCAGTGGGTG |
| | GATATGACAGATGTGACTGTCTGGCTCAAT |
| | TCCACACATGCATCAAAAACATGTAAACCA |
| | GCATTGGGCTACAGTTTTGCAGCTGGCACT |
| | ATTGATGGAGTTGGAGGCCTCAATTTTACA |
| | CAGGGGAAAACAGAAGGGGATCCAMTTGGG |
| | ACACCATTCGGGACCAGATCCTGGGAAAGC |
| | CATCTGAAGAAATTAAAGAATGTCATAAAC |
| | CAAAGCCCATCCTTCTTCACACCGGAGAAC |
| | TATCAAAACCTCACCCCTGGCATCCAGACA |
| | TTGTTGATGTTCAGATTATTACCCTTGGGT |
| | CCTTGGCCATAACTGCCATCCCCGGGGAGT |
| | TTACGACCATGTCTGGACGAAGACTTCGAG |
| | AGGCAGTTCAAGCAGAATTTGCATCTCATG |
| | GGATGCAGAACATGACTGTTGTTATTTCAG |
| | GTCTATGCAACGTCTATACACATTACATTA |
| | CCACTTATGAAGAATACCAGGCTCAGCGAT |
| | ATGAGGCAGCATCGACAATTTATGGACCGC |
| | ACACATTATCTGCTTACATTCAGCTCTTCA |
| | GAAACCTTGCTAAGGCTATTGCTACGGACA |
| | CGGTAGCCAACCTGAGCAGAGGTCCAGAAC |
| | CTCCCTTTTTCAAACAATTAATAGTTCCAT |
| | TAATTCCTAGTATTGTGGATAGAGCACCAA |
| | AAGGCAGAACTTTCGGGGATGTCCTGCAGC |
| | CAGCAAAACCTGAATACAGAGTGGGGGAAG |
| | TTGCTGAAGTTATATTTGTAGGTGCTAACC |
| | CGAAGAATTCAGTACAAAACCAGACCCATC |
| | AGACCTTCCTCACTGTGGAGAAATATGAGG |
| | CTACTTCAACATCGTGGCAGATAGTGTGTA |
| | ATGATGCCTCCTGGGAGACTCGTTTTTATT |
| | GGCACAAGGGACTCCTGGGTCTGAGTAATG |
| | CAACAGTGGAATGGCATATTCCAGACACTG |
| | CCCAGCCTGGAATCTACAGAATAAGATATT |
| | TTGGACACAATCGGAAGCAGGACATTCTGA |
| | AGCCTGCTGTCATACTTTCATTTGAAGGCA |
| | CTTCCCCGGCTTTTGAAGTTGTAACTATTT |
| | AGTGA |
| | (SEQ ID NO: 8) |
| ASAH2 | ATGGCCAAACGCACCTTCTCTAACTTGGAG |
| transcript | ACATTCCTGATTTTCCTCCTTGTAATGATG |
| variant 2 | AGTGCCATCACAGTGGCCCTTCTCAGCCTC |
| | TTGTTTATCACCAGTGGGACCATTGAAAAC |
| | CACAAAGATTTAGGAGGCCATTTTTTTTCA |
| | ACCACCCAAAGCCCTCCAGCCACCCAGGGC |
| | TCCACAGCTGCCCAACGCTCCACAGCCACC |
| | CAGCATTCCACAGCCACCCAGAGCTCCACA |
| | GCCACTCAAACTTCTCCAGTGCCTTTAACC |
| | CCAGAGTCTCCTCTATTTCAGAACTTCAGT |
| | GGCTACCATATTGGTGTTGGACGAGCTGAC |
| | TGCACAGGACAAGTAGCAGATATCAATTTG |
| | ATGGGCTATGGCAAATCCGGCCAGAATGCA |
| | CAGGGCATCCTCACCAGGCTATACAGTCGT |
| | GCCTTCATCATGGCAGAACCTGATGGGTCC |
| | AATCGAACAGTGTTTGTCAGCATCGACATA |
| | GGCATGGTATCACAAAGGCTCAGGCTGGAG |
| | GTCCTGAACAGAC |
| | TGCAGAGTAAATATGGCTCCCTGTACAGAA |
| | GAGATAATGTCATCCTGAGTGGCACTCACA |
| | CTCATTCAGGTCCTGCAGGATATTTCCAGT |
| | ATACCGTGTTTGTAATTGCCAGTGAAGGAT |
| | TTAGCAATCAAACTTTTCAGCACATGGTCA |
| | CTGGTATCTTGAAGAGCATTGACATAGCAC |
| | ACACAAATATGAAACCAGGCAAAATCTTCA |
| | TCAATAAAGGAAATGTGGATGGTGTGCAGA |
| | TCAACAGAAGTCCGTATTCTTACCTTCAAA |
| | ATCCGCAGTCAGAGAGAGCAAGGTATTCTT |
| | CAAATACAGACAAGGAAATGATAGTTTTGA |
| | AAATGGTAGATTTGAATGGAGATGACTTGG |
| | GCCTTATCAGCTGGTTTGCCATCCACCCGG |
| | TCAGCATGAACAACAGTAACCATCTTGTAA |
| | ACAGTGACAATGTGGGCTATGCATCTTACC |
| | TGCTTGAGCAAGAGAAGAACAAAGGATATC |
| | TACCTGGACAGGGGCCATTTGTAGCAGCCT |
| | TTGCTTCATCAAACCTAGGAGATGTGTCCC |
| | CCAACATTCTTGGACCACGTTGCATCAACA |
| | CAGGAGAGTCCTGTGATAACGCCAATAGCA |
| | CTTGTCCCATTGGTGGGCCTAGCATGTGCA |
| | TTGCTAAGGGACCTGGACAGGATATGTTTG |
| | ACAGCACACAAATTATAGGACGGGCCATGT |
| | ATCAGAGAGCAAAGTCAAAAACATGTAAAC |
| | CAGCATTGGGCTACAGTTTTGCAGCTGGCA |
| | CTATTGATGGAGTTGGAGGCCTCAATTTTA |
| | CACAGGGGAAAACAGAAGGGGATCCATTTT |
| | GGGACACCATTCGGGACCAGATCCTGGGAA |
| | AGCCATCTGAAGAAATTAAAGAATGTCATA |
| | AACCAAAGCCCATCCTTCTTCACACCGGAG |
| | AACTATCAAAACCTCACCCCTGGCATCCAG |
| | ACATTGTTGATGTTCAGATTATTACCCTTG |
| | GGTCCTTGGCCATAACTGCCATCCCCGGGG |
| | AGTTTACGACCATGTCTGGACGAAGACTTC |
| | GAGAGGCAGTTCAAGCAGAATTTGCATCTC |
| | ATGGGATGCAGAACATGACTGTTGTTATTT |
| | CAGGTCTATGCAACGTCTATACACATTACA |
| | TTACCACTTATGAAGAATACCAGGCTCAGC |
| | GATATGAGGCAGCATCGACAATTTATGGAC |
| | CGCACACATTATCTGCTTACATTCAGCTCT |
| | TCAGAAACCTTGCTAAGGCTATTGCTACGG |
| | ACACGGTAGCCAACCTGAGCAGAGGTCCAG |
| | AACCTCCCTTTTTCAAACAATTAATAGTTC |
| | CATTAATTCCTAGTATTGTGGATAGAGCAC |
| | CAAAAGGCAGAACTTTCGGGGATGTCCTGC |
| | AGCCAGCAAAACCTGAATACAGAGTGGGGG |
| | AAGTTGCTGAAGTTATATTTGTAGGTGCTA |
| | ACCCGAAGAATTCAGTACAAAACCAGACCC |
| | ATCAGACCTTCCTCACTGTGGAGAAATATG |
| | AGGCTACTTCAACATCGTGGCAGATAGTGT |
| | GTAATGATGCCTCCTGGGAGACTCGTTTTT |
| | TATTGGCACAAGGGACTCCTGGGTCTGAGT |
| | AATGCAACAGTGGAATGGCATATTCCAGAC |
| | ACTGCCCAGCCTGGAATCTACAGAATAAGA |
| | TATTTTGGACACAATCGGAAGCAGGACATT |
| | CTGAAGCCTGCTGTCATACTTTCATTTGAA |
| | GGCACTTCCCCGGCTTTTGAAGTTGTAACT |
| | ATTTAGTGA |
| | (SEQ ID NO: 9) |
| ASAH2B | ATGAGGCAGCATCGACAATTTATGGACCGC |
| transcript | ACGCATTATCTGCTTACATTCAGCTCTTCA |
| variant 1 | GAAACCTTGCTAAGGCTATTGCTACGTATT |
| | GTGGATAGAGCACCAAAAGGCAGAACTTTC |
| | GGGGATGTCCTGCAGCCAGCAAAACCTGAA |
| | TACAGAGTGGGGGAAGTTGCTGAAGTTATA |
| | TTTGTAGGTGCTAACCCGAAGAATTCAGTA |
| | CAAAACCAGACCCATCAGACCTTCCTCACT |
| | GTGGAGAAATATGAGGCTACTTCAACATCG |
| | TGGCAGATAGTGTGTAATGATGCCTCCTGG |
| | GAGACTCGTTTTTATTGGCACAAGGGACTC |
| | CTGGGTCTGAGTAATGCAACAGTGGAATGG |
| | CATATTCCAGACACTGCCCAGCCTGGAATC |
| | TACAGAATAAGATATTTTGGACACAATCGG |
| | AAGCAGGACATTCTGAAGCCTGCTGTCATA |
| | CTTTCATTTGAAGGCACTTCCCCGGCTTTT |
| | GAAGTTGTAACTATTTAGTGA |
| | (SEQ ID NO: 10) |
| ASAH2B | ATGGTAGCCAACCTGAGCAGAGGTCCAGAA |
| transcript | CCTCCCTTTTTCAAACAATTAATAGTTCCA |
| variant 3 | TTAATTCCTAGTATTGTGGATAGAGCACCA |
| | AAAGGCAGAACTTTCGGGGATGTCCTGCAG |
| | CCAGCAAAACCTGAATACAGAGTGGGGGAA |
| | GTTGCTGAAGTTATATTTGTAGGTGCTAAC |
| | CCGAAGAATTCAGTACAAAACCAGACCCAT |
| | CAGACCTTCCTCACTGTGGAGAAATATGAG |
| | GCTACTTCAACATCGTGGCAGATAGTGTGT |
| | AATGATGCCTCCTGGGAGACTCGTTTTTAT |
| | TGGCACAAGGGACTCCTGGGTCTGAGTAAT |
| | GCAACAGTGGAATGGCATATTCCAGACACT |
| | GCCCAGCCTGGAATCTACAGAATAAGATAT |
| | TTTGGACACAATCGGAAGCAGGACATTCTG |
| | AAGCCTGCTGTCATACTTTCATTTGAAGGC |
| | ACTTCCCCGGCTTTTGAAGTTGTAACTATT |
| | TAGTGAATGGTAGCCAACCTGAGCAGAGGT |
| | CCAGAACCTCCCTTTTTCAAACAATTAATA |
| | GTTCCATTAATTCCTAGTATTGTGGATAGA |
| | GCACCAAAAGGCAGAACTTTCGGGGATGTC |
| | CTGCAGCCAGCAAAACCTGAATACAGAGTG |
| | GGGGAAGTTGCTGAAGTTATATTTGTAGGT |
| | GCTAACCCGAAGAATTCAGTACAAAACCAG |
| | ACCCATCAGACCTTCCTCACTGTGGAGAAA |
| | TATGAGGCTACTTCAACATCGTGGCAGATA |
| | GTGTGTAATGATGCCTCCTGGGAGACTCGT |
| | TTTTATTGGCACAAGGGACTCCTGGGTCTG |
| | AGTAATGCAACAGTGGAATGGCATATTCCA |
| | GACACTGCCCAGCCTGGAATCTACAGAATA |
| | AGATATTTTGGACACAATCGGAAGCAGGAC |
| | ATTCTGAAGCCTGCTGTCATACTTTCATTT |
| | GAAGGCACTTCCCCGGCTTTTGAAGTTGTA |
| | ACTATTTAGTGA |
| | (SEQ ID NO: 11) |
| ASAH2B | ATGGTAGCCAACCTGAGCAGAGGTCCAGAA |
| transcript | CCTCCCTTTTTCAAACAATTAATAGTTCCA |
| variant 4 | TTAATTCCTAGTATTGTGGATAGAGCACCA |
| | AAAGGCAGAACTTTCGGGGATGTCCTGCAG |
| | CCAGCAAAACCTGAATACAGAGTGGGGGAA |
| | GTTGCTGAAGTTATATTTGTAGGTGCTAAC |
| | CCGAAGAATTCAGTACAAAACCAGACCCAT |
| | CAGACCTTCCTCACTGTGGAGAAATATGAG |
| | GCTACTTCAACATCGTGGCAGATAGTGTGT |
| | AATGATGCCTCCTGGGAGACTCGTTTTTAT |
| | TGGCACAAGGGACTCCTGGGTCTGAGTAAT |
| | GCAACAGTGGAATGGCATATTCCAGACACT |
| | GCCCAGCCTGGAATCTACAGAATAAGATAT |
| | TTTGGACACAATCGGAAGCAGGACATTCTG |
| | AAGCCTGCTGTCATACTTTCATTTGAAGGC |
| | ACTTCCCCGGCTTTTGAAGTTGTAACTATT |
| | TAG |
| | (SEQ ID NO: 12) |
| ACER1 | ATGCCTAGCATCTTCGCCTATCAGAGCTCC |
| | GAGGTGGACTGGTGTGAGAGCAACTTCCAG |
| | TACTCGGAGCTGGTGGCCGAGTTCTACAAC |
| | ACGTTCTCCAATATCCCCTTCTTCATCTTC |
| | GGGCCACTGATGATGCTCCTGATGCACCCG |
| | TATGCCCAGAAGCGCTCCCGCTACATTTAC |
| | GTTGTCTGGGTCCTCTTCATGATCATAGGC |
| | CTGTTCTCCATGTATTTCCACATGACGCTC |
| | AGCTTCCTGGGCCAGCTGCTGGACGAGATC |
| | GCCATCCTGTGGCTCCTGGGCAGTGGCTAT |
| | AGCATATGGATGCCCCGCTGCTATTTCCCC |
| | TCCTTCCTTGGGGGGAACAGGTCCCAGTTC |
| | ATCCGCCTGGTCTTCATCACCACTGTGGTC |
| | AGCACCCTTCTGTCCTTCCTGCGGCCCACG |
| | GTCAACGCCTACGCCCTCAACAGCATTGCC |
| | CTGCACATTCTCTACATCGTGTGCCAGGAG |
| | TACAGGAAGACCAGCAATAAGGAGCTTCGG |
| | CACCTGATTGAGGTCTCCGTGGTTTTATGG |
| | GCTGTTGCTCTGACCAGCTGGATCAGTGAC |
| | CGTCTGCTTTGCAGCTTCTGGCAGAGGATT |
| | CATTTCTTCTATCTGCACAGCATCTGGCAT |
| | GTGCTCATCAGCATCACCTTCCCTTATGGC |
| | ATGGTCACCATGGCCTTGGTGGATGCCAAC |
| | TATGAGATGCCAGGTGAAACCCTCAAAGTC |
| | CGCTACTGGCCTCGGGACAGTTGGCCCGTG |
| | GGGCTGCCCTACGTGGAAATCCGGGGTGAT |
| | GACAAGGACTGCTGA |
| | (SEQ ID NO: 13) |
| ACER2 | ATGGGCGCCCCGCACTGGTGGGACCAGCTG |
| | CAGGCTGGTAGCTCGGAGGTGGACTGGTGC |
| | GAGGACAACTACACCATCGTGCCTGCTATC |
| | GCCGAGTTCTACAACACGATCAGCAATGTC |
| | TTATTTTTCATMTACCGCCCATCTGCATGT |
| | GCTTGTTTCGTCAGTATGCAACATGCTTCA |
| | ACAGTGGCATCTACTTAATCTGGACTCTTT |
| | TGGTTGTAGTGGGAATTGGATCCGTCTACT |
| | TCCATGCAACCCTTAGTTTCTTGGGTCAGA |
| | TGCTTGATGAACTTGCAGTCCTTTGGGTTC |
| | TGATGTGTGCTTTGGCCATGTGGTTCCCCA |
| | GAAGGTATCTACCAAAGATCTTTCGGAATG |
| | ACCGGGGTAGGTTCAAGGTGGTGGTCAGTG |
| | TCCTGTCTGCGGTTACGACGTGCCTGGCAT |
| | TTGTCAAGCCTGCCATCAACAACATCTCTC |
| | TGATGACCCTGGGAGTTCCTTGCACTGCAC |
| | TGCTCATCGCAGAGCTAAAGAGGTGTGACA |
| | ACATGCGTGTGTTTAAGCTGGGCCTCTTCT |
| | CGGGCCTCTGGTGGACCCTGGCCCTGTTCT |
| | GCTGGATCAGTGACCGAGCTTTCTGCGAGC |
| | TGCTGTCATCCTTCAACTTCCCCTACCTGC |
| | ACTGCATGTGGCACATCCTCATCTGCCTTG |
| | CTGCCTACCTGGGCTGTGTATGCTTTGCCT |
| | ACTTTGATGCTGCCTCAGAGATTCCTGAGC |
| | AAGGCCCTGTCATCAAGTTCTGGCCCAATG |
| | AGAAATGGGCCTTCATTGGTGTCCCCTATG |
| | TGTCCCTCCTGTGTGCCAACAAGAAATCAT |
| | CAGTCAAGATCACGTGA |
| | (SEQ ID NO: 14) |
| ACER3 | ATGGCTCCGGCCGCGGACCGAGAGGGCTAC |
| transcript | TGGGGCCCCACGACCTCCACGCTGGACTGG |
| variant 1 | TGCGAGGAGAACTACTCCGTGACCTGGTAC |
| | ATCGCCGAGTTCTGGAATACAGTGAGTAAC |
| | CTGATCATGATTATACCTCCAATGTTCGGT |
| | GCAGTTCAGAGTGTTAGAGACGGTCTGGAA |
| | AAGCGGTACATTGCTTCTTATTTAGCACTC |
| | ACAGTGGTAGGAATGGGATCCTGGTGCTTC |
| | CACATGACTCTGAAATATGAAATGCAGCTA |
| | TTGGATGAACTCCCAATGATATACAGCTGT |
| | TGCATATTTGTGTACTGCATGTTTGAATGT |
| | TTCAAGATCAAGAACTCAGTAAACTACCAT |
| | CTGCTTTTTACCTTAGTTCTATTCAGTTTA |
| | ATAGTAACCACAGTTTACCTTAAGGTAAAA |
| | GAGCCGATATTCCATCAGGTCATGTATGGA |
| | ATGTTGGTCTTTACATTAGTACTTCGATCT |
| | ATTTATATTGTTACATGGGTTTATCCATGG |
| | CTTAGAGGACTGGGTTATACATCATTGGGT |
| | ATATTTMATTGGGATTTTTATTTTGGAATA |
| | TAGATAACATATTTTGTGAGTCACTGAGGA |
| | ACTTTCGAAAGAAGGTACCACCTATCATAG |
| | GTATTACCACACAATTTCATGCATGGTGGC |
| | ATATTTTAACTGGCCTTGGTTCCTATCTTC |
| | ACATCCTTTTCAGTTTGTATACAAGAACAC |
| | TTTACCTGAGATATAGGCCAAAAGTGAAGT |
| | TTCTCTTTGGAATCTGGCCAGTGATCCTGT |
| | TTGAGCCTCTCAGGAAGCATVGA |
| | (SEQ ID NO: 15) |
| ACER3 | ATGGCTCCGGCCGCGGACCGAGAGGGCTAC |
| transcript | TGGGGCCCCACGACCTCCACGCTGGACTGG |
| variant 2 | TGCGAGGAGAACTACTCCGTGACCTGGTAC |
| | ATCGCCGAGTTCTTGGTAGGAATGGGATCC |
| | TGGTGCTTCCACATGACTCTGAAATATGAA |
| | ATGCAGCTATTGGATGAACTCCCAATGATA |
| | TACAGCTGTTGCATATTTGTGTACTGCATG |
| | TTTGAATGTTTCAAGATCAAGAACTCAGTA |
| | AACTACCATCTGCTTTTTACCTTAGTTCTA |
| | TTCAGTTTAATAGTAACCACAGTTTACCTT |
| | AAGGTAAAAGAGCCGATATTCCATCAGGTC |
| | ATGTATGGAATGTTGGTCTTTACATTAGTA |
| | CTTCGATCTATTTATATTGTTACATGGGTT |
| | TATCCATGGCTTAGAGGACTGGGTTATACA |
| | TCATTGGGTATATTTTTATTGGGATTTTTA |
| | TTTTGGAATATAGATAACATATTTTGTGAG |
| | TCACTGAGGAACTTTCGAAAGAAGGTACCA |
| | CCTATCATAGGTATTACCACACAATTTCAT |
| | GCATGGTGGCATATTTTAACTGGCCTTGGT |
| | TCCTATCTTCACATCCTTTTCAGTTTGTAT |
| | ACAAGAACACTTTACCTGAGATATAGGCCA |
| | AAAGTGAAGTTTCTCTTTGGAATCTGGCCA |
| | GTGATCCTGTTTGAGCCTCTCAGGAAGCAT |
| | GA(SEQIDN0:16) |
| ACER3 | ATGATATACAGCTGTTGCATATTTGTGTAC |
| transcript | TGCATGTTTGAATGTTTCAAGATCAAGAAC |
| variant 3 | TCAGTAAACTACCATCTGCTTTTTACCTTA |
| | GTTCTATTCAGTTTAATAGTAACCACAGTT |
| | TACCTTAAGGTAAAAGAGCCGATATTCCAT |
| | CAGGTCATGTATGGAATGTTGGTCTTTACA |
| | TTAGTACTTCGATCTATTTATATTGTTACA |
| | TGGGTTTATCCATGGCTTAGAGGACTGGGT |
| | TATACATCATTGGGTATATTTTTATTGGGA |
| | TTTTTATTTTGGAATATAGATAACATATTT |
| | TGTGAGTCACTGAGGAACTTTCGAAAGAAG |
| | GTACCACCTATCATAGGTATTACCACACAA |
| | TTTCATGCATGGTGGCATATTTTAACTGGC |
| | CTTGGTTCCTATCTTCACATCCTTTTCAGT |
| | TTGTATACAAGAACACTTTACCTGAGATAT |
| | AGGCCAAAAGTGAAGTTTCTCTTTGGAATC |
| | TGGCCAGTGATCCTGTTTGAGCCTCTCAGG |
| | AAGCATTGA |
| | (SEQ ID NO: 17) |
| Sphk2 | ATGAATGGACACCTTGAAGCAGAGGAGCAG |
| | CAGGACCAGAGGCCAGACCAGGAGCTGACC |
| | GGGAGCTGGGGCCACGGGCCTAGGAGCACC |
| | CTGGTCAGGGCTAAGGCCATGGCCCCGCCC |
| | CCACCGCCACTGGCTGCCAGCACCCCGCTC |
| | CTCCATGGCGAGTTTGGCTCCTACCCAGCC |
| | CGAGGCCCACGCTTTGCCCTCACCCTTACA |
| | TCGCAGGCCCTGCACATACAGCGGCTGCGC |
| | CCCAAACCTGAAGCCAGGCCCCGGGGTGGC |
| | CTGGTCCCGTTGGCCGAGGTCTCAGGCTGC |
| | TGCACCCTGCGAAGCCGCAGCCCCTCAGAC |
| | TCAGCGGCCTACTTCTGCATCTACACCTAC |
| | CCTCGGGGCCGGCGCGGGGCCCGGCGCAGA |
| | GCCACTCGCACCTTCCGGGCAGATGGGGCC |
| | GCCACCTACGAAGAGAACCGTGCCGAGGCC |
| | CAGCGCTGGGCCACTGCCCTCACCTGTCTG |
| | CTCCGAGGACTGCCACTGCCCGGGGATGGG |
| | GAGATCACCCCTGACCTGCTACCTCGGCCG |
| | CCCCGGTTGCTTCTATTGGTCAATCCCTTT |
| | GGGGGTCGGGGCCTGGCCTGGCAGTGGTGT |
| | AAGAACCACGTGCTTCCCATGATCTCTGAA |
| | GCTGGGCTGTCCTTCAACCTCATCCAGACA |
| | GAACGACAGAACCACGCCCGGGAGCTGGTC |
| | CAGGGGCTGAGCCTGAGTGAGTGGGATGGC |
| | ATCGTCACGGTCTCGGGAGACGGGCTGCTC |
| | CATGAGGTGCTGAACGGGCTCCTAGATCGC |
| | CCTGACTGGGAGGAAGCTGTGAAGATGCCT |
| | GTGGGCATCCTCCCCTGCGGCTCGGGCAAC |
| | GCGCTGGCCGGAGCAGTGAACCAGCACGGG |
| | GGATTTGAGCCAGCCCTGGGCCTCGACCTG |
| | TTGCTCAACTGCTCACTGTTGCTGTGCCGG |
| | GGTGGTGGCCACCCACTGGACCTGCTCTCC |
| | GTGACGCTGGCCTCGGGCTCCCGCTGTTTC |
| | TCCTTCCTGTCTGTGGCCTGGGGCTTCGTG |
| | TCAGATGTGGATATCCAGAGCGAGCGCTTC |
| | AGGGCCTTGGGCAGTGCCCGCTTCACACTG |
| | GGCACGGTGCTGGGCCTCGCCACACTGCAC |
| | ACCTACCGCGGACGCCTCTCCTACCTCCCC |
| | GCCACTGTGGAACCTGCCTCGCCCACCCCT |
| | GCCCATAGCCTGCCTCGTGCCAAGTCGGAG |
| | CTGACCCTAACCCCAGACCCAGCCCCGCCC |
| | ATGGCCCACTCACCCCTGCATCGTTCTGTG |
| | TCTGACCTGCCTCTTCCCCTGCCCCAGCCT |
| | GCCCTGGCCTCTCCTGGCTCGCCAGAACCC |
| | CTGCCCATCCTGTCCCTCAACGGTGGGGGC |
| | CCAGAGCTGGCTGGGGACTGGGGTGGGGCT |
| | GGGGATGCTCCGCTGTCCCCGGACCCACTG |
| | CTGTCTTCACCTCCTGGCTCTCCCAAGGCA |
| | GCTCTACACTCACCCGTCTCCGAAGGGGCC |
| | CCCGTAATTCCCCCATCCTCTGGGCTCCCA |
| | CTTCCCACCCCTGATGCCCGGGTAGGGGCC |
| | TCCACCTGCGGCCCGCCCGACCACCTGCTG |
| | CCTCCGCTGGGCACCCCGCTGCCCCCAGAC |
| | TGGGTGACGCTGGAGGGGGACTTTGTGCTC |
| | ATGTTGGCCATCTCGCCCAGCCACCTAGGC |
| | GCTGACCTGGTGGCAGCTCCGCATGCGCGC |
| | TTCGACGACGGCCTGGTGCACCTGTGCTGG |
| | GTGCGTAGCGGCATCTCGCGGGCTGCGCTG |
| | CTGCGCCTTTTCTTGGCCATGGAGCGTGGT |
| | AGCCACTTCAGCCTGGGCTGTCCGCAGCTG |
| | GGCTACGCCGCGGCCCGTGCCTTCCGCCTA |
| | GAGCCGCTCACACCACGCGGCGTGCTCACA |
| | GTGGACGGGGAGCAGGTGGAGTATGGGCCG |
| | CTACAGGCACAGATGCACCCTGGCATCGGT |
| | ACACTGCTCACTGGGCCTCCTGGCTGCCCG |
| | GGGCGGGAGCCCTGA |
| | (SEQ ID NO: 18) |
| CerK | ATGGGGGCGACGGGGGCGGCGGAGCCGCTG |
| | CAATCCGTGCTGTGGGTGAAGCAGCAGCGC |
| | TGCGCCGTGAGCCTGGAGCCCGCGCGGGCT |
| | CTGCTGCGCTGGTGGCGGAGCCCGGGGCCC |
| | GGAGCCGGCGCCCCCGGCGCGGATGCCTGC |
| | TCTGTGCCTGTATCTGAGATCATCGCCGTT |
| | GAGGAAACAGACGTTCACGGGAAACATCAA |
| | GGCAGTGGAAAATGGCAGAAAATGGAAAAG |
| | CCTTACGCTTTTACAGTTCACTGTGTAAAG |
| | AGAGCACGACGGCACCGCTGGAAGTGGGCG |
| | CAGGTGACTTTCTGGTGTCCAGAGGAGCAG |
| | CTGTGTCACTTGTGGCTGCAGACCCTGCGG |
| | GAGATGCTGGAGAAGCTGACGTCCAGACCA |
| | AAGCATTTACTGGTATTTATCAACCCGTTT |
| | GGAGGAAAAGGACAAGGCAAGCGGATATAT |
| | GAAAGAAAAGTGGCACCACTGTTCACCTTA |
| | GCCTCCATCACCACTGACATCATCGTTACT |
| | GAACATGCTAATCAGGCCAAGGAGACTCTG |
| | TATGAGATTAACATAGACAAATACGACGGC |
| | ATCGTCTGTGTCGGCGGAGATGGTATGTTC |
| | AGCGAGGTGCTGCACGGTCTGATTGGGAGG |
| | ACGCAGAGGAGCGCCGGGGTCGACCAGAAC |
| | CACCCCCGGGCTGTGCTGGTCCCCAGTAGC |
| | CTCCGGATTGGAATCATTCCCGCAGGGTCA |
| | ACGGACTGCGTGTGTTACTCCACCGTGGGC |
| | ACCAGCGACGCAGAAACCTCGGCGCTGCAT |
| | ATCGTTGTTGGGGACTCGCTGGCCATGGAT |
| | GTGTCCTCAGTCCACCACAACAGCACACTC |
| | CTTCGCTACTCCGTGTCCCTGCTGGGCTAC |
| | GGCTTCTACGGGGACATCATCAAGGACAGT |
| | GAGAAGAAACGGTGGTTGGGTCTTGCCAGA |
| | TACGACTTTTCAGGTTTAAAGACCTTCCTC |
| | TCCCACCACTGCTATGAAGGGACAGTGTCC |
| | TTCCTCCCTGCACAACACACGGTGGGATCT |
| | CCAAGGGATAGGAAGCCCTGCCGGGCAGGA |
| | TGCTTTGTTTGCAGGCAAAGCAAGCAGCAG |
| | CTGGAGGAGGAGCAGAAGAAAGCACTGTAT |
| | GGTTTGGAAGCTGCGGAGGACGTGGAGGAG |
| | TGGCAAGTCGTCTGTGGGAAGTTTCTGGCC |
| | ATCAATGCCACAAACATGTCCTGTGCTTGT |
| | CGCCGGAGCCCCAGGGGCCTCTCCCCGGCT |
| | GCCCACTTGGGAGACGGGTCTTCTGACCTC |
| | ATCCTCATCCGGAAATGCTCCAGGTTCAAT |
| | TTTCTGAGATTTCTCATCAGGCACACCAAC |
| | CAGCAGGACCAGTTTGACTTCACTTTTGTT |
| | GAAGTTTATCGCGTCAAGAAATTCCAGTTT |
| | ACGTCGAAGCACATGGAGGATGAGGACAGC |
| | GACCTCAAGGAGGGGGGGAAGAAGCGCTTT |
| | GGGCACATTTGCAGCAGCCACCCCTCCTGC |
| | TGCTGCACCGTCTCCAACAGCTCCTGGAAC |
| | TGCGACGGGGAGGTCCTGCACAGCCCTGCC |
| | ATCGAGGTCAGAGTCCACTGCCAGCTGGTT |
| | CGACTCTTTGCACGAGGAATTGAAGAGAAT |
| | CCGAAGCCAGACTCACACAGCTGA |
| | (SEQ ID NO: 19) |

### EXAMPLES

### Mice

All animal procedures were performed under protocols approved by the Icahn School of Medicine at Mount Sinai Institutional Care and Use Committee.

### Synthesis of Anc80.A C

The nucleotide sequence for an embodiment of the Anc80 plasmid described herein is shown below. A map of the vector is also shown in FIG. 16.

### Anc80 Plasmid Sequence

| | |
|---|---|
| pAAV.CMV. | CTGCGCGCTCGCTCGCTCACTGAGGCCGCC |
| WPRE.bGH.d | CGGGCAAAGCCCGGGCGTCGGGCGACCTTT |
| na | GGTCGCCCGGCCTCAGTGAGCGAGCGAGCG |
| | CGCAGAGAGGGAGTGGCCAACTCCATCACT |
| | AGGGGTTCCTTGTAGTTAATGATTAACCCG |
| | CCATGCTACTTATCTACGTAGCCATGCTCT |
| | AGGAAGATCGGAATTCGCCCTTAAGCTAGC |
| | TAGTTATTAATAGTAATCAATTACGGGGTC |
| | ATTAGTTCATAGCCCATATATGGAGTTCCG |
| | CGTTACATAACTTACGGTAAATGGCCCGCC |
| | TGGCTGACCGCCCAACGACCCCCGCCCATT |
| | GACGTCAATAATGACGTATGTTCCCATAGT |
| | AACGCCAATAGGGACTTTCCATTGACGTCA |
| | ATGGGTGGAGTATTTACGGTAAACTGCCCA |
| | CTTGGCAGTACATCAAGTGTATCATATGCC |
| | AAGTACGCCCCCTATTGACGTCAATGACGG |
| | TAAATGGCCCGCCTGGCATTATGCCCAGTA |
| | CATGACCTTATGGGACTTTCCTACTTGGCA |
| | GTACATCTACGTATTAGTCATCGCTATTAC |
| | CATGGTGATGCGGTTTTGGCAGTACATCAA |
| | TGGGCGTGGATAGCGGTTTGACTCACGGGG |
| | ATTTCCAAGTCTCCACCCCATTGACGTCAA |
| | TGGGAGTTTGTTTTGGCACCAAAATCAACG |
| | GGACTTTCCAAAATGTCGTAACAACTCCGC |
| | CCCATTGACGCAAATGGGCGGTAGGCGTGT |
| | ACGGTGGGAGGTCTATATAAGCAGAGCTGG |
| | TTTAGTGAACCGTCAGATCCTGCAGAAGTT |
| | GGTCGTGAGGCACTGGGCAGGTAAGTATCA |
| | AGGTTACAAGACAGGTTTAAGGAGACCAAT |
| | AGAAACTGGGCTTGTCGAGACAGAGAAGAC |
| | TCTTGCGTTTCTGATAGGCACCTATTGGTC |
| | TTACTGACATCCACTTTGCCTTTCTCTCCA |
| | CAGGTGTCCAGGCGGCCGCNNNGGATCCAA |
| | TCAACCTCTGGATTACAAAATTTGTGAAAG |
| | ATTGACTGGTATTCTTAACTATGTTGCTCC |
| | TTTTACGCTATGTGGATACGCTGCTTTAAT |
| | GCCTTTGTATCATGCTATTGCTTCCCGTAT |
| | GGCTTTCATTTTCTCCTCCTTGTATAAATC |
| | CTGGTTGCTGTCTCTTTATGAGGAGTTGTG |
| | GCCCGTTGTCAGGCAACGTGGCGTGGTGTG |
| | CACTGTGTTTGCTGACGCAACCCCCACTGG |
| | TTGGGGCATTGCCACCACCTGTCAGCTCCT |
| | TTCCGGGACTTTCGCTTTCCCCCTCCCTAT |
| | TGCCACGGCGGAACTCATCGCCGCCTGCCT |
| | TGCCCGCTGCTGGACAGGGGCTCGGCTGTT |
| | GGGCACTGACAATTCCGTGGTGTTGTCGGG |
| | GAAATCATCGTCCTTTCCTTGGCTGCTCGC |
| | CTGTGTTGCCACCTGGATTCTGCGCGGGAC |
| | GTCCTTCTGCTACGTCCCTTCGGCCCTCAA |
| | TCCAGCGGACCTTCCTTCCCGCGGCCTGCT |
| | GCCGGCTCTGCGGCCTCTTCCGCGTCTTCG |
| | AGATCTGCCTCGACTGTGCCTTCTAGTTGC |
| | CAGCCATCTGTTGTTTGCCCCTCCCCCGTG |
| | CCTTCCTTGACCCTGGAAGGTGCCACTCCC |
| | ACTGTCCTTTCCTAATAAAATGAGGAAATT |
| | GCATCGCATTGTCTGAGTAGGTGTCATTCT |
| | ATTCTGGGGGGTGGGGTGGGGCAGGACAGC |
| | AAGGGGGAGGATTGGGAAGACAATAGCAGG |
| | CATGCTGGGGACTCGAGTTAAGGGCGAATT |
| | CCCGATAAGGATCTTCCTAGAGCATGGCTA |
| | CGTAGATAAGTAGCATGGCGGGTTAATCAT |
| | TAACTACAAGGAACCCCTAGTGATGGAGTT |
| | GGCCACTCCCTCTCTGCGCGCTCGCTCGCT |
| | CACTGAGGCCGGGCGACCAAAGGTCGCCCG |
| | ACGCCCGGGCTTTGCCCGGGCGGCCTCAGT |
| | GAGCGAGCGAGCGCGCAGCCTTAATTAACC |
| | TAATTCACTGGCCGTCGTTTTACAACGTCG |
| | TGACTGGGAAAACCCTGGCGTTACCCAACT |
| | TAATCGCCTTGCAGCACATCCCCCTTTCGC |
| | CAGCTGGCGTAATAGCGAAGAGGCCCGCAC |
| | CGATCGCCCTTCCCAACAGTTGCGCAGCCT |
| | GAATGGCGAATGGGACGCGCCCTGTAGCGG |
| | CGCATTAAGCGCGGCGGGTGTGGTGGTTAC |
| | GCGCAGCGTGACCGCTACACTTGCCAGCGC |
| | CCTAGCGCCCGCTCCTTTCGCTTTCTTCCC |
| | TTCCTTTCTCGCCACGTTCGCCGGCTTTCC |
| | CCGTCAAGCTCTAAATCGGGGGCTCCCTTT |
| | AGGGTTCCGATTTAGTGCTTTACGGCACCT |
| | CGACCCCAAAAAACTTGATTAGGGTGATGG |
| | TTCACGTAGTGGGCCATCGCCCTGATAGAC |
| | GGTTTTTCGCCCTTTGACGTTGGAGTCCAC |
| | GTTCTTTAATAGTGGACTCTTGTTCCAAAC |
| | TGGAACAACACTCAACCCTATCTCGGTCTA |
| | TTCTTTTGATTTATAAGGGATTTTGCCGAT |
| | TTCGGCCTATTGGTTAAAAAATGAGCTGAT |
| | TTAACAAAAATTTAACGCGAATTTTAACAA |
| | AATATTAACGTTTATAATTTCAGGTGGCAT |
| | CTTTCGGGGAAATGTGCGCGGAACCCCTAT |
| | TTGTTTATTTTTCTAAATACATTCAAATAT |
| | GTATCCGCTCATGAGACAATAACCCTGATA |
| | AATGCTTCAATAATATTGAAAAAGGAAGAG |
| | TATGAGTATTCAACATTTCCGTGTCGCCCT |
| | TATTCCCTTTTTTGCGGCATTTTGCCTTCC |
| | TGTTTTTGCTCACCCAGAAACGCTGGTGAA |
| | AGTAAAAGATGCTGAAGATCAGTTGGGTGC |
| | ACGAGTGGGTTACATCGAACTGGATCTCAA |
| | TAGTGGTAAGATCCTTGAGAGTTTTCGCCC |
| | CGAAGAACGTTTTCCAATGATGAGCACTTT |
| | TAAAGTTCTGCTATGTGGCGCGGTATTATC |
| | CCGTATTGACGCCGGGCAAGAGCAACTCGG |
| | TCGCCGCATACACTATTCTCAGAATGACTT |
| | GGTTGAGTACTCACCAGTCACAGAAAAGCA |
| | TCTTACGGATGGCATGACAGTAAGAGAATT |
| | ATGCAGTGCTGCCATAACCATGAGTGATAA |
| | CACTGCGGCCAACTTACTTCTGACAACGAT |
| | CGGAGGACCGAAGGAGCTAACCGCTTTTTT |
| | GCACAACATGGGGGATCATGTAACTCGCCT |
| | TGATCGTTGGGAACCGGAGCTGAATGAAGC |
| | CATACCAAACGACGAGCGTGACACCACGAT |
| | GCCTGTAGTAATGGTAACAACGTTGCGCAA |
| | ACTATTAACTGGCGAACTACTTACTCTAGC |
| | TTCCCGGCAACAATTAATAGACTGGATGGA |
| | GGCGGATAAAGTTGCAGGACCACTTCTGCG |
| | CTCGGCCCTTCCGGCTGGCTGGTTTATTGC |
| | TGATAAATCTGGAGCCGGTGAGCGTGGGTC |
| | TCGCGGTATCATTGCAGCACTGGGGCCAGA |
| | TGGTAAGCCCTCCCGTATCGTAGTTATCTA |
| | CACGACGGGGAGTCAGGCAACTATGGATGA |
| | ACGAAATAGACAGATCGCTGAGATAGGTGC |
| | CTCACTGATTAAGCATTGGTAACTGTCAGA |
| | CCAAGTTTACTCATATATACTTTAGATTGA |
| | TTTAAAACTTCATTTTTAATTTAAAAGGAT |
| | CTAGGTGAAGATCCTTTTTGATAATCTCAT |
| | GACCAAAATCCCTTAACGTGAGTTTTCGTT |
| | CCACTGAGCGTCAGACCCCGTAGAAAAGAT |
| | CAAAGGATCTTCTTGAGATCCTTTTTTTCT |
| | GCGCGTAATCTGCTGCTTGCAAACAAAAAA |
| | ACCACCGCTACCAGCGGTGGTTTGTTTGCC |
| | GGATCAAGAGCTACCAACTCTTTTTCCGAA |
| | GGTAACTGGCTTCAGCAGAGCGCAGATACC |
| | AAATACTGTCCTTCTAGTGTAGCCGTAGTT |
| | AGGCCACCACTTCAAGAACTCTGTAGCACC |
| | GCCTACATACCTCGCTCTGCTAATCCTGTT |
| | ACCAGTGGCTGCTGCCAGTGGCGATAAGTC |
| | GTGTCTTACCGGGTTGGACTCAAGACGATA |
| | GTTACCGGATAAGGCGCAGCGGTCGGGCTG |
| | AACGGGGGGTTCGTGCACACAGCCCAGCTT |
| | GGAGCGAACGACCTACACCGAACTGAGATA |
| | CCTACAGCGTGAGCTATGAGAAAGCGCCAC |
| | GCTTCCCGAAGGGAGAAAGGCGGACAGGTA |
| | TCCGGTAAGCGGCAGGGTCGGAACAGGAGA |
| | GCGCACGAGGGAGCTTCCAGGGGGAAACGC |
| | CTGGTATCTTTATAGTCCTGTCGGGTTTCG |
| | CCACCTCTGACTTGAGCGTCGATTTTTGTG |
| | ATGCTCGTCAGGGGGGCGGAGCCTATGGAA |
| | AAACGCCAGCAACGCGGCCTTTTTACGGTT |
| | CCTGGCCTTTTGCTGCGGTTTTGCTCACAT |
| | GTTCTTTCCTGCGTTATCCCCTGATTCTGT |
| | GGATAACCGTATTACCGCCTTTGAGTGAGC |
| | TGATACCGCTCGCCGCAGCCGAACGACCGA |
| | GCGCAGCGAGTCAGTGAGCGAGGAAGCGGA |
| | AGAGCGCCCAATACGCAAACCGCCTCTCCC |
| | CGCGCGTTGGCCGATTCATTAATGCAGCTG |
| | GCACGACAGGTTTCCCGACTGGAAAGCGGG |
| | CAGTGAGCGCAACGCAATTAATGTGAGTTA |
| | GCTCACTCATTAGGCACCCCAGGCTTTACA |
| | CTTTATGCTTCCGGCTCGTATGTTGTGTGG |
| | AATTGTGAGCGGATAACAATTTCACACAGG |
| | AAACAGCTATGACCATGATTACGCCAGATT |
| | TAATTAAGG (SEQ ID NO: 20) |

Total RNA was isolated using the RNeasy mini kit (QIAGEN) and reverse transcribed using Superscript III reverse transcriptase (Invitrogen), according to the manufacturer's instructions. Real-time qPCR analyses were performed on a Mastercycler realplex 4 Sequence Detector (Eppendoff) using SYBR Green (Quantitect™ SYBR Green PCR Kit, QIAGEN). Data were normalized to 18srRNA expression where appropriate (endogenous controls). Fold changes of gene expression were determined by the ddCT method. PCR primer sequences are summarized in Table 2.

TABLE 2

| | | SEQ | | SEQ |
|---|---|---|---|---|
| | | ID | | ID |
| Gene | Forward | NO. | Reverse | NO. |
| AC | ACAGGATTCA | 21 | TGGGCATCTT | 22 |
| | AACCAGGACT | | TCCTTCCGAA | |
| | GT | | | |
| AC | TGACAGGATT | 23 | CTGGGCATCT | 24 |
| | CAAACCAGGA | | TTCCTTCCGA | |
| | CT | | | |
| Sphk1 | ATACTCACCG | 25 | CCATTAGCCC | 26 |
| | AACGGAAGAA | | ATTCACCACC | |
| | CC | | TC | |
| Sphk1 | ACTGATACTC | 27 | CATTAGCCCA | 28 |
| | ACCGAACGGA | | TTCACCACCT | |
| | A | | C | |
| S1PR2 | CACAGCCAAC | 29 | TCTGAGTATA | 30 |
| | AGTCTCCAAA | | AGCCGCCCA | |
| S1PR2 | ATAGACCGAG | 31 | GAACCTTCTC | 32 |
| | CACAGCCAA | | AGGATTGAGG | |
| | | | T | |
| 18s rRNA* | TAACGAACGA | 33 | CGGACATCTA | 34 |
| | GACTCTGGCA | | AGGGCATCAC | |
| | T | | AG | |

*Genetic Vaccines and Therapy 2004, 2:5

### Western Blot

Upon thawing, hearts lysates' were subjected to separation by SDS-PAGE using 12% precast Nupage Bis/Tris gels (Invitrogen, Carlsbad, Calif., USA) under reducing conditions and MES running buffer (Invitrogen), and transferred onto a nitrocellulose membrane (Bio-Rad) using a semidry transfer apparatus and Nupage-MOPS transfer buffer (Invitrogen). The membrane was block with TBS/Tween containing 5% dry milk and incubated with specific primary antibodies over night at 4° C. washed with TBS/Tween and incubated with rabbit or goat antibodies conjugated to horseradish peroxidase for 1 hour at room temperature. Detection was performed by an enhanced chemiluminecence (ECL) detection system (Pierce, Rockford, Ill.). For molecular weight determination prestained protein standards (Amersham, Buckinghamshire, UK) were used.

### Immunohistochemistry

The mouse hearts were harvested and perfused using perfusion buffer (2 g/I butanedione, monoxime and 7.4 g/I KCI in PBSx1) and 4% paraformaldehyde (PFA). Hearts were fixed in 4% PFA/PBS overnight on shaker and then washed with PBS for 1 hr and incubated in 30% sucrose/PBS at 40 C overnight. Before freezing, hearts were mounted in OCT for 30 min and frozen at −80° C. Transverse heart sections of 10 μM were made by cryostat. Cryosections were washed in PBST and blocked for 1 h with 5% donkey serum in PBST. Sections were incubated over night at 4° C. using primary antibodies for Troponin I, Sphk1, S1p2. Secondary antibodies were used for fluorescent labeling (Jackson ImmunoResearch Laboratories). TUNEL staining was performed according to manufacturer's recommendations (In-Situ Cell Death Detection Kit, Fluorescein, Cat# 11684795910, Roche). Stained sections were imaged using a Zeiss Slide Scanner Axio Scan or Zeiss mic. Quantification of TUNEL in cardiac sections was performed using ImageJ software. For cell immunocytochemistry, Hek293 and isolated CMs were fixed on coverslips with 4% PFA for 10 min at room temperature. Following permeabilization with 0.1% TRITON® X100 in PBS for 10 min at room temperature, cells were blocked with 5% Donkey serum+0.1% TRITON® X100 in PBS for 30 minutes. Coverslips were incubated with primary antibodies in humidity chamber for 1 hour at room temperature followed by incubation with corresponding secondary antibodies conjugated to Alexa Fluor 488, Alexa Fluor 647 and Alexa Fluor 555, and Hoechst 33342 staining for nuclei visualization (all from Invitrogene). The fluorescent images were taken on a Zeiss fluorescent microscope at 20× magnification.

Model of PAH A rat PAH model was used. Pneumonectomy combined with Sugen rat model results in fast pulmonary vascular remodeling comparable to clinical PAH and development of the plexiform lesions found in human PAH. AC gene was introduced using Anc80 as viral vector to the lung via intratracheal transfer.

### Cardiovascular Evaluation

MRI was used to assess the effect of Anc80-AC on heart function and PAH parameters (right ventricular hemodynamics including ejection fraction, hypertrophy, pulmonary artery pressure and vascular resistance).

### Tissue Evaluation

Animal tissues from Sprague-Dawley rats will be analyzed for RNA sequencing, proteomics and sphingolipids quantification.

Study groups: 1. No Anc80/AC no PAH; 2. Saline +PAH; 3. Anc80 only+PAH; 4. Anc80/AC, No PAH; 5. Anc80/AC+PAH.

### Preliminary Results

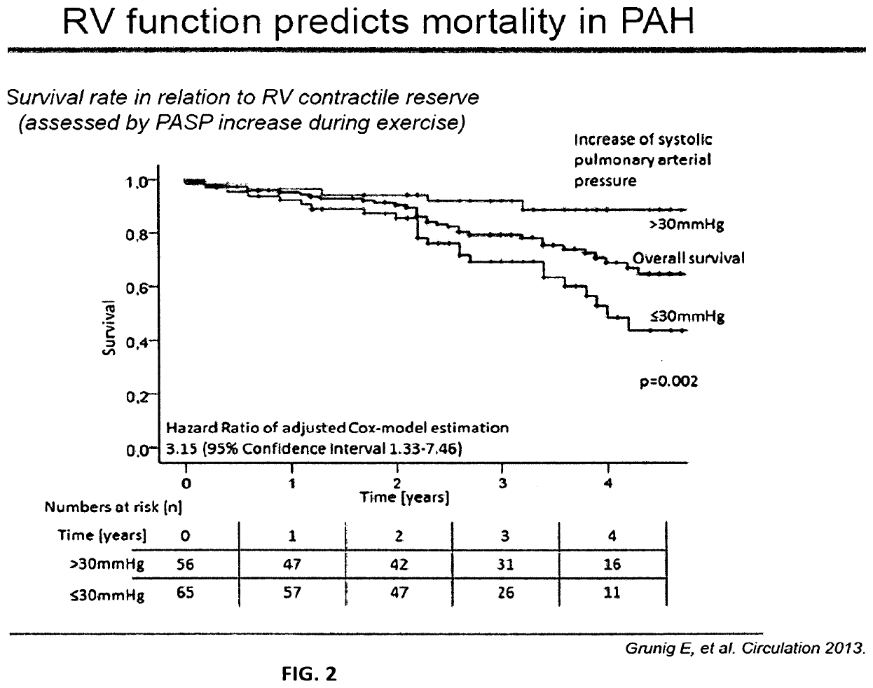

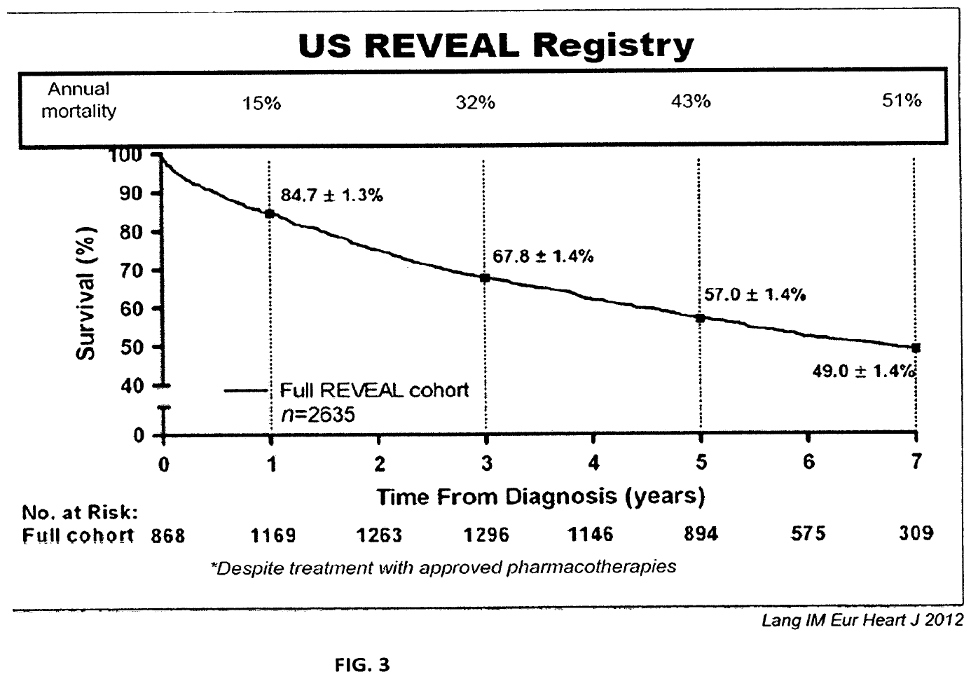

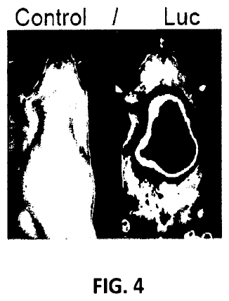

Rats were subjected to PAH induction protocol (FIG. 2). At week 0, rats were subjected to baseline MRI, RV and PA catheterization to measure the pressure, followed by left lung removal. On day 7 pneumonectomized rats were subjected to SU5416 (Su/Pn 10 mg/kg) administration (SC injection). Induced animals demonstrated severely elevated mean PA pressures and developed neointima and smooth muscle hypertrophy (FIGS. 3 and 4). On week 4 PAH induced rats were treated with Anc80 AC (1×10¹¹genome copies). On weeks 6 and 8 animals were validated by MRI for heart function and RV and PA catheterization for pressure measurement.

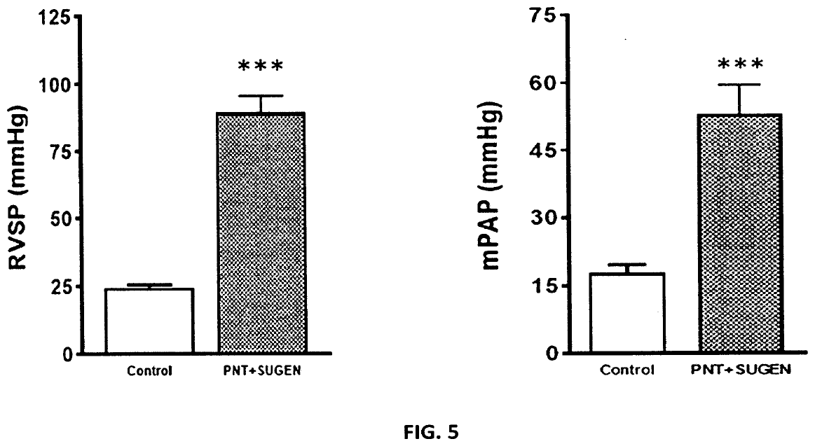

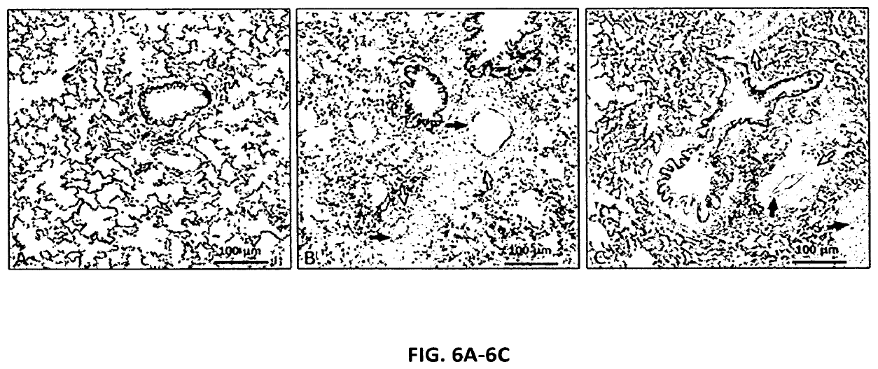

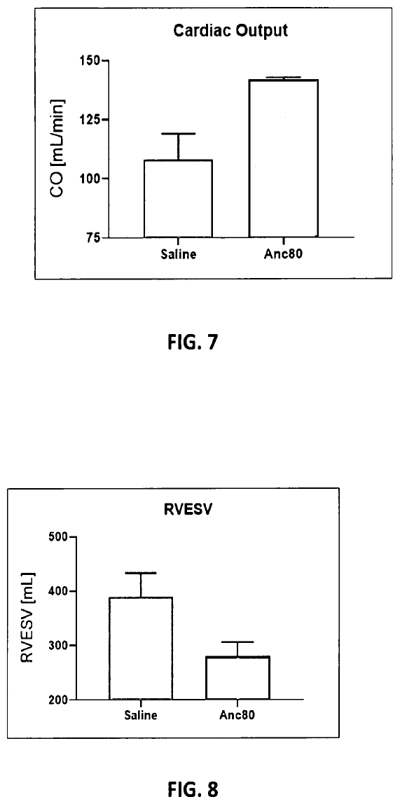

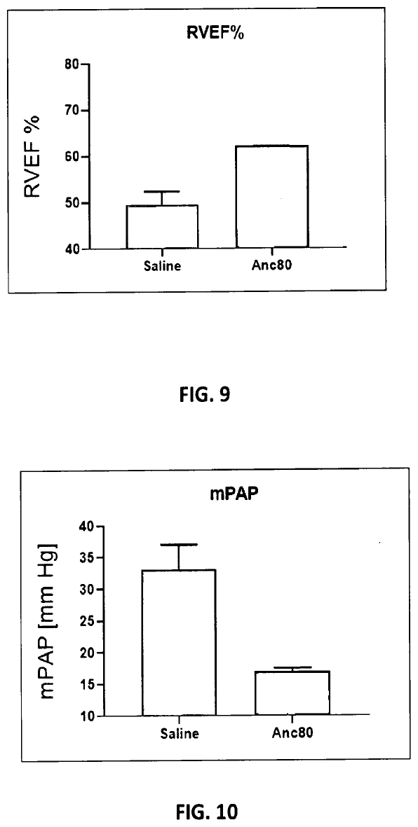

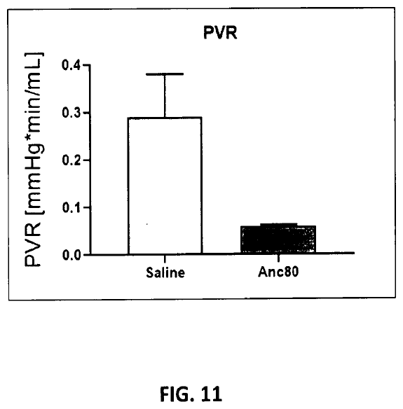

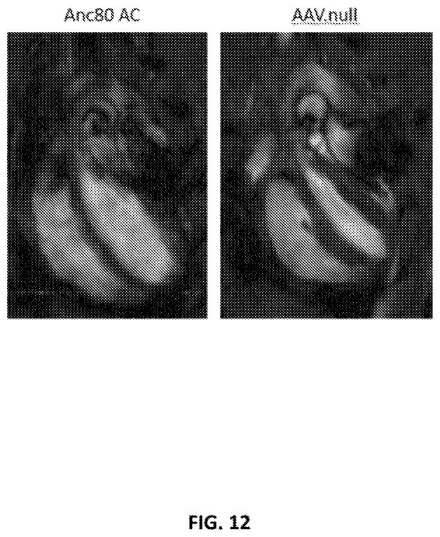

Preliminary PAH results with AC-Anc80 gene therapy were outstanding (see FIGS. 5-10). In control animals a severe PAH develops after SU5416 (Su/Pn) administration to pneumonectomized rats. Induced animals demonstrated severely elevated mean PA pressures and developed neointima and smooth muscle hypertrophy. After AC administration cardiac output increased in 32% (FIG. 5), right ventricular systolic volume decreased in 39% (FIG. 6), right ventricular ejection fraction increased in 65% (FIG. 7), mean pulmonary artery pressure decreased in 94% (FIG. 8) and mean pulmonary vascular resistance decreased in 4.8 times (FIG. 9). Animals treated with AC Anc80 at 8 weeks showed excellent cardiac function (validated by MRI, FIG. 10) and normal PA pressures despite PAH disease present. In one embodiment, ancestral 80 (Anc80) viral vector was used for gene delivery. Anc80 has been used as a viral vector with low immunogenicity and ancestral strains that are not regularly recognized by human antibodies, unlike common AAVs that are seropositive in >50% of the population. Anc80 delivery has provided rapid onset of expression in less than 48 hours. The Anc80 virus demonstrated the ability to generate very high transduction of lung and other cardiovascular tissues. Overall, the Anc80 viral vector provided an ideal delivery vehicle for the gene (FIG. 17).

It is to be understood that, while the methods and compositions of matter have been described herein in conjunction with a number of different aspects, the forgoing description of the various aspects is intended to illustrate and not limit the scope of the methods and compositions of matter. Other aspects, advantages, and modifications are within the scope of the following claims.

### REFERENCES

Perez G I, Tao X J, Tilly J L. Fragmentation and death (a.k.a. apoptosis) of ovulated oocytes. Mol Hum Reprod. 1999;5(5):414-20.

Eliyahu E, Park J H, Shtraizent N, He X, Schuchman E H. Acid ceramidase is a novel factor required for early embryo survival. FASEB J. 2007;21(7):1403-9.

Eliyahu E, Shtraizent N, Martinuzzi K, Barritt J, He X, Wei H, Chaubal S, Copperman A B, Schuchman E H. Acid ceramidase improves the quality of oocytes and embryos and the outcome of in vitro fertilization. FASEB J. 2010;24(4):1229-38.

Katalin Karikó, Hiromi Muramatsu, Frank A Welsh, János Ludwig, Hiroki Kato, Shizuo Akira, Drew Weissman. Incorporation of Pseudouridine Into mRNA Yields Superior Nonimmunogenic Vector With Increased Translational Capacity and Biological Stability. Mol Ther. 2008;16(11): 1833-1840.

Yang H, Wang H, Shivalila C S, Cheng A W, Shi L, Jaenisch R. One-step generation of mice carrying reporter and conditional alleles by CRISPR/Cas-mediated genome engineering. Cell. 2013;154(6):1370-9.

Wu Y, Liang D, Wang Y, Bai M, Tang W, Bao S, Yan Z, Li D, Li J. Correction of a genetic disease in mouse via use of CRISPR-Cas9. Cell Stem Cell. 2013;13(6):659-62.

Ruzo A, Brivanlou A H. At Last: Gene Editing in Human Embryos to Understand Human Development. Cell Stem Cell. 2017;21(5):564-565.

Frumkin T, Peleg S, Gold V, Reches A, Asaf S, Azem F, Ben-Yosef D, Malcov M. Complex chromosomal rearrangement-a lesson learned from PGS. J Assist Reprod Genet. 2017; 34(8): 1095-1100.

Zinn et al. In Silico Reconstruction of the Viral Evolutionary Lineage Yields a Potent Gene Therapy Vector, Cell Reports 12. 1056-1068 (2015)

1. A method to minimize damage to a cell/group of cells/tissue in a subject as the result of disease or injury comprising administering to the subject a therapeutically effective amount of an Anc80 viral vector that codes for the expression of a sphingolipid-metabolizing protein.

2. The method of claim 1, wherein the disease is selected from the group consisting of pulmonary arterial hypertension (PAH), stroke, ischemia and reperfusion injury.

3. The method of claim 1, wherein the damage is to pulmonary tissue as the result of pulmonary arterial hypertension (PAH).

4. The method of claim 1, wherein the damage is to cardiac tissue as the result of pulmonary arterial hypertension (PAH).

5. The method of claim 1, wherein said sphingolipid-metabolizing protein is selected from (1) a ceramidase; (2) sphingosine kinase (SPHK); (3) sphingosine-1-phosphate receptor (SIPR); (4) ceramidase kinase (CERK); or a combination of any of (1), (2), (3) and (4).

6. The method of claim 1, wherein said sphingolipid-metabolizing protein is an acid ceramidase.

7. The method of claim 1, wherein said sphingolipid-metabolizing protein is a neutral ceramidase.

8. The method of claim 1, wherein said sphingolipid-metabolizing protein is an alkaline ceramidase.

9. The method of claim 1, wherein the sphingolipid-metabolizing protein is a ceramidase encoded by a nucleic acid selected from the group consisting of SEQ ID NO: 1, SEQ ID NO: 6, SEQ ID NO: 7, SEQ ID NO: 8, SEQ ID NO: 9, SEQ ID NO: 10, SEQ ID NO: 11, and SEQ ID NO: 12.

10. The method of claim 1, wherein Anc80 has the nucleotide sequence of SEQ ID NO: 20.

11. A pharmaceutical composition comprising an Anc80 viral vector that codes for expression of a sphingolipid-metabolizing protein and a pharmaceutically acceptable carrier.

12. The pharmaceutical composition of claim 11, wherein said sphingolipid-metabolizing protein is selected from the group consisting of (1) a ceramidase; (2) sphingosine kinase (SPHK); (3) sphingosine-1-phosphate receptor (SIPR); and any combination of 1, 2 and 3.

13. The pharmaceutical composition of claim 11, wherein said sphingolipid-metabolizing protein is an acid ceramidase.

14. The pharmaceutical composition of claim 11, wherein said sphingolipid-metabolizing protein is a neutral ceramidase.

15. The pharmaceutical composition of claim 11, wherein said sphingolipid-metabolizing protein is an alkaline ceramidase.

16. An Anc80 viral vector that codes for expression of a sphingolipid-metabolizing protein for use in the mitigation of tissue damage resulting from disease or injury.

17. The Anc80 viral vector of claim 16, wherein the disease is selected from the group consisting of pulmonary arterial hypertension (PAH), stroke, ischemia and reperfusion injury.

18. The Anc80 viral vector of claim 16 for use in the mitigation of tissue damage from pulmonary arterial hypertension (PAH).

19. The Anc80 viral vector of claim 16, wherein the tissue is lung tissue.

20. The Anc80 viral vector of claim 16, wherein the tissue is cardiac tissue.

21. The Anc80 viral vector of claim 16, wherein said sphingolipid-metabolizing protein is acid ceramidase.

22.-28. (canceled)


SEQUENCE LISTING (34 sequences)

SEQ ID NO: 1 - DNA, 1188 bp, Artificial Sequence
  synthetic vector
atgccgggccggagttgcgtcgccttagtcctcctggctgccgccgtcagctgtgccgtcgcgcagcacgcgccgccgtggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtga

SEQ ID NO: 2 - DNA, 1197 bp, Artificial Sequence
  synthetic oligomer
atggatccagtggtcggttgcggacgtggcctctttggttttgttttctcagcgggcggcccccggggcgtgctcccgcggccctgccgcgtgctggtgctgctgaacccgcgcggcggcaagggcaaggccttgcagctcttccggagtcacgtgcagccccttttggctgaggctgaaatctccttcacgctgatgctcactgagcggcggaaccacgcgcgggagctggtgcggtcggaggagctgggccgctgggacgctctggtggtcatgtctggagacgggctgatgcacgaggtggtgaacgggctcatggagcggcctgactgggagaccgccatccagaagcccctgtgtagcctcccagcaggctctggcaacgcgctggcagcttccttgaaccattatgctggctatgagcaggtcaccaatgaagacctcctgaccaactgcacgctattgctgtgccgccggctgctgtcacccatgaacctgctgtctctgcacacggcttcggggctgcgcctcttctctgtgctcagcctggcctggggcttcattgctgatgtggacctagagagtgagaagtatcggcgtctgggggagatgcgcttcactctgggcaccttcctgcgtctggcagccctgcgcacctaccgcggccgactggcctacctccctgtaggaagagtgggttccaagacacctgcctcccccgttgtggtccagcagggcccggtagatgcacaccttgtgccactggaggagccagtgccctctcactggacagtggtgcccgacgaggactttgtgctagtcctggcactgctgcactcgcacctgggcagtgagatgtttgctgcacccatgggccgctgtgcagctggcgtcatgcatctgttctacgtgcgggcgggagtgtctcgtgccatgctgctgcgcctcttcctggccatggagaagggcaggcatatggagtatgaatgcccctacttggtatatgtgcccgtggtcgccttccgcttggagcccaaggatgggaaaggtgtgtttgcagtggatggggaattgatggttagcgaggccgtgcagggccaggtgcacccaaactacttctggatggtcagcggttgcgtggagcccccgcccagctggaagccccagcagatgccaccgccagaagagcccttatga

SEQ ID NO: 3 - DNA, 1062 bp, Artificial Sequence
  synthetic oligomer
atgggcagcttgtactcggagtacctgaaccccaacaaggtccaggaacactataattataccaaggagacgctggaaacgcaggagacgacctcccgccaggtggcctcggccttcatcgtcatcctctgttgcgccattgtggtggaaaaccttctggtgctcattgcggtggcccgaaacagcaagttccactcggcaatgtacctgtttctgggcaacctggccgcctccgatctactggcaggcgtggccttcgtagccaataccttgctctctggctctgtcacgctgaggctgacgcctgtgcagtggtttgcccgggagggctctgccttcatcacgctctcggcctctgtcttcagcctcctggccatcgccattgagcgccacgtggccattgccaaggtcaagctgtatggcagcgacaagagctgccgcatgcttctgctcatcggggcctcgtggctcatctcgctggtcctcggtggcctgcccatccttggctggaactgcctgggccacctcgaggcctgctccactgtcctgcctctctacgccaagcattatgtgctgtgcgtggtgaccatcttctccatcatcctgttggccatcgtggccctgtacgtgcgcatctactgcgtggtccgctcaagccacgctgacatggccgccccgcagacgctagccctgctcaagacggtcaccatcgtgctaggcgtctttatcgtctgctggctgcccgccttcagcatcctccttctggactatgcctgtcccgtccactcctgcccgatcctctacaaagcccactactttttcgccgtctccaccctgaattccctgctcaaccccgtcatctacacgtggcgcagccgggacctgcggcgggaggtgcttcggccgctgcagtgctggaggccgggggtgggggtgcaaggacggaggcggggcgggaccccgggccaccacctcctgccactccgcagctccagctccctggagaggggcatgcacatgcccacgtcacccacgtttctggagggcaacacggtggtcatg

SEQ ID NO: 4 - DNA, 1653 bp, Artificial Sequence
  synthetic oligomer
atggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaa

SEQ ID NO: 5 - DNA, 801 bp, Artificial Sequence
  synthetic oligomer
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggagatccaaaaaagaagagaaaggtaggcgatccaaaaaagaagagaaaggtaggtgatccaaaaaagaagagaaaggtataa

SEQ ID NO: 6 - DNA, 1236 bp, Artificial Sequence
  synthetic oligomer
atgaactgctgcatcgggctgggagagaaagctcgcgggtcccaccgggcctcctacccaagtctcagcgcgcttttcaccgaggcctcaattctgggatttggcagctttgctgtgaaagcccaatggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtga

SEQ ID NO: 7 - DNA, 1170 bp, Artificial Sequence
  synthetic oligomer
atgaactgctgcatcgggctgggagagaaagctcgcgggtcccaccgggcctcctacccaagtctcagcgcgcttttcaccgaggcctcaattctgggatttggcagctttgctgtgaaagcccaatggacagaggactgcagaaaatcaacctatcctccttcaggaccaactgtcttccctgctgttataaggtacagaggtgcagttccatggtacaccataaatcttgacttaccaccctacaaaagatggcatgaattgatgcttgacaaggcaccagtgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtga

SEQ ID NO: 8 - DNA, 2346 bp, Artificial Sequence
  synthetic oligomer
atggccaaacgcaccttctctaacttggagacattcctgattttcctccttgtaatgatgagtgccatcacagtggcccttctcagcctcttgtttatcaccagtgggaccattgaaaaccacaaagatttaggaggccattttttttcaaccacccaaagccctccagccacccagggctccacagctgcccaacgctccacagccacccagcattccacagccacccagagctccacagccactcaaacttctccagtgcctttaaccccagagtctcctctatttcagaacttcagtggctaccatattggtgttggacgagctgactgcacaggacaagtagcagatatcaatttgatgggctatggcaaatccggccagaatgcacagggcatcctcaccaggctatacagtcgtgccttcatcatggcagaacctgatgggtccaatcgaacagtgtttgtcagcatcgacataggcatggtatcacaaaggctcaggctggaggtcctgaacagactgcagagtaaatatggctccctgtacagaagagataatgtcatcctgagtggcactcacactcattcaggtcctgcaggatatttccagtataccgtgtttgtaattgccagtgaaggatttagcaatcaaacttttcagcacatggtcactggtatcttgaagagcattgacatagcacacacaaatatgaaaccaggcaaaatcttcatcaataaaggaaatgtggatggtgtgcagatcaacagaagtccgtattcttaccttcaaaatccgcagtcagagagagcaaggtattcttcaaatacagacaaggaaatgatagttttgaaaatggtagatttgaatggagatgacttgggccttatcagctggtttgccatccacccggtcagcatgaacaacagtaaccatcttgtaaacagtgacaatgtgggctatgcatcttacctgcttgagcaagagaagaacaaaggatatctacctggacaggggccatttgtagcagcctttgcttcatcaaacctaggagatgtgtcccccaacattcttggaccacgttgcatcaacacaggagagtcctgtgataacgccaatagcacttgtcccattggtgggcctagcatgtgcattgctaagggacctggacaggatatgtttgacagcacacaaattataggacgggccatgtatcagagagcaaaggaactctatgcctctgcctcccaggaggtaacaggaccactggcttcagcacaccagtgggtggatatgacagatgtgactgtctggctcaattccacacatgcatcaaaaacatgtaaaccagcattgggctacagttttgcagctggcactattgatggagttggaggcctcaattttacacaggggaaaacagaaggggatccattttgggacaccattcgggaccagatcctgggaaagccatctgaagaaattaaagaatgtcataaaccaaagcccatccttcttcacaccggagaactatcaaaacctcacccctggcatccagacattgttgatgttcagattattacccttgggtccttggccataactgccatccccggggagtttacgaccatgtctggacgaagacttcgagaggcagttcaagcagaatttgcatctcatgggatgcagaacatgactgttgttatttcaggtctatgcaacgtctatacacattacattaccacttatgaagaataccaggctcagcgatatgaggcagcatcgacaatttatggaccgcacacattatctgcttacattcagctcttcagaaaccttgctaaggctattgctacggacacggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtga

SEQ ID NO: 9 - DNA, 2241 bp, Artificial Sequence
  synthetic oligomer
atggccaaacgcaccttctctaacttggagacattcctgattttcctccttgtaatgatgagtgccatcacagtggcccttctcagcctcttgtttatcaccagtgggaccattgaaaaccacaaagatttaggaggccattttttttcaaccacccaaagccctccagccacccagggctccacagctgcccaacgctccacagccacccagcattccacagccacccagagctccacagccactcaaacttctccagtgcctttaaccccagagtctcctctatttcagaacttcagtggctaccatattggtgttggacgagctgactgcacaggacaagtagcagatatcaatttgatgggctatggcaaatccggccagaatgcacagggcatcctcaccaggctatacagtcgtgccttcatcatggcagaacctgatgggtccaatcgaacagtgtttgtcagcatcgacataggcatggtatcacaaaggctcaggctggaggtcctgaacagactgcagagtaaatatggctccctgtacagaagagataatgtcatcctgagtggcactcacactcattcaggtcctgcaggatatttccagtataccgtgtttgtaattgccagtgaaggatttagcaatcaaacttttcagcacatggtcactggtatcttgaagagcattgacatagcacacacaaatatgaaaccaggcaaaatcttcatcaataaaggaaatgtggatggtgtgcagatcaacagaagtccgtattcttaccttcaaaatccgcagtcagagagagcaaggtattcttcaaatacagacaaggaaatgatagttttgaaaatggtagatttgaatggagatgacttgggccttatcagctggtttgccatccacccggtcagcatgaacaacagtaaccatcttgtaaacagtgacaatgtgggctatgcatcttacctgcttgagcaagagaagaacaaaggatatctacctggacaggggccatttgtagcagcctttgcttcatcaaacctaggagatgtgtcccccaacattcttggaccacgttgcatcaacacaggagagtcctgtgataacgccaatagcacttgtcccattggtgggcctagcatgtgcattgctaagggacctggacaggatatgtttgacagcacacaaattataggacgggccatgtatcagagagcaaagtcaaaaacatgtaaaccagcattgggctacagttttgcagctggcactattgatggagttggaggcctcaattttacacaggggaaaacagaaggggatccattttgggacaccattcgggaccagatcctgggaaagccatctgaagaaattaaagaatgtcataaaccaaagcccatccttcttcacaccggagaactatcaaaacctcacccctggcatccagacattgttgatgttcagattattacccttgggtccttggccataactgccatccccggggagtttacgaccatgtctggacgaagacttcgagaggcagttcaagcagaatttgcatctcatgggatgcagaacatgactgttgttatttcaggtctatgcaacgtctatacacattacattaccacttatgaagaataccaggctcagcgatatgaggcagcatcgacaatttatggaccgcacacattatctgcttacattcagctcttcagaaaccttgctaaggctattgctacggacacggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtga

SEQ ID NO: 10 - DNA, 501 bp, Artificial Sequence
  synthetic oligomer
atgaggcagcatcgacaatttatggaccgcacgcattatctgcttacattcagctcttcagaaaccttgctaaggctattgctacgtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtga

SEQ ID NO: 11 - DNA, 972 bp, Artificial Sequence
  synthetic oligomer
atggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtgaatggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtga

SEQ ID NO: 12 - DNA, 483 bp, Artificial Sequence
  synthetic oligomer
atggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttag

SEQ ID NO: 13 - DNA, 795 bp, Artificial Sequence
  synthetic oligomer
atgcctagcatcttcgcctatcagagctccgaggtggactggtgtgagagcaacttccagtactcggagctggtggccgagttctacaacacgttctccaatatccccttcttcatcttcgggccactgatgatgctcctgatgcacccgtatgcccagaagcgctcccgctacatttacgttgtctgggtcctcttcatgatcataggcctgttctccatgtatttccacatgacgctcagcttcctgggccagctgctggacgagatcgccatcctgtggctcctgggcagtggctatagcatatggatgccccgctgctatttcccctccttccttggggggaacaggtcccagttcatccgcctggtcttcatcaccactgtggtcagcacccttctgtccttcctgcggcccacggtcaacgcctacgccctcaacagcattgccctgcacattctctacatcgtgtgccaggagtacaggaagaccagcaataaggagcttcggcacctgattgaggtctccgtggttttatgggctgttgctctgaccagctggatcagtgaccgtctgctttgcagcttctggcagaggattcatttcttctatctgcacagcatctggcatgtgctcatcagcatcaccttcccttatggcatggtcaccatggccttggtggatgccaactatgagatgccaggtgaaaccctcaaagtccgctactggcctcgggacagttggcccgtggggctgccctacgtggaaatccggggtgatgacaaggactgctga

SEQ ID NO: 14 - DNA, 828 bp, Artificial Sequence
  synthetic oligomer
atgggcgccccgcactggtgggaccagctgcaggctggtagctcggaggtggactggtgcgaggacaactacaccatcgtgcctgctatcgccgagttctacaacacgatcagcaatgtcttatttttcattttaccgcccatctgcatgtgcttgtttcgtcagtatgcaacatgcttcaacagtggcatctacttaatctggactcttttggttgtagtgggaattggatccgtctacttccatgcaacccttagtttcttgggtcagatgcttgatgaacttgcagtcctttgggttctgatgtgtgctttggccatgtggttccccagaaggtatctaccaaagatctttcggaatgaccggggtaggttcaaggtggtggtcagtgtcctgtctgcggttacgacgtgcctggcatttgtcaagcctgccatcaacaacatctctctgatgaccctgggagttccttgcactgcactgctcatcgcagagctaaagaggtgtgacaacatgcgtgtgtttaagctgggcctcttctcgggcctctggtggaccctggccctgttctgctggatcagtgaccgagctttctgcgagctgctgtcatccttcaacttcccctacctgcactgcatgtggcacatcctcatctgccttgctgcctacctgggctgtgtatgctttgcctactttgatgctgcctcagagattcctgagcaaggccctgtcatcaagttctggcccaatgagaaatgggccttcattggtgtcccctatgtgtccctcctgtgtgccaacaagaaatcatcagtcaagatcacgtga

SEQ ID NO: 15 - DNA, 804 bp, Artificial Sequence
  synthetic oligomer
atggctccggccgcggaccgagagggctactggggccccacgacctccacgctggactggtgcgaggagaactactccgtgacctggtacatcgccgagttctggaatacagtgagtaacctgatcatgattatacctccaatgttcggtgcagttcagagtgttagagacggtctggaaaagcggtacattgcttcttatttagcactcacagtggtaggaatgggatcctggtgcttccacatgactctgaaatatgaaatgcagctattggatgaactcccaatgatatacagctgttgcatatttgtgtactgcatgtttgaatgtttcaagatcaagaactcagtaaactaccatctgctttttaccttagttctattcagtttaatagtaaccacagtttaccttaaggtaaaagagccgatattccatcaggtcatgtatggaatgttggtctttacattagtacttcgatctatttatattgttacatgggtttatccatggcttagaggactgggttatacatcattgggtatatttttattgggatttttattttggaatatagataacatattttgtgagtcactgaggaactttcgaaagaaggtaccacctatcataggtattaccacacaatttcatgcatggtggcatattttaactggccttggttcctatcttcacatccttttcagtttgtatacaagaacactttacctgagatataggccaaaagtgaagtttctctttggaatctggccagtgatcctgtttgagcctctcaggaagcattga

SEQ ID NO: 16 - DNA, 693 bp, Artificial Sequence
  synthetic oligomer
atggctccggccgcggaccgagagggctactggggccccacgacctccacgctggactggtgcgaggagaactactccgtgacctggtacatcgccgagttcttggtaggaatgggatcctggtgcttccacatgactctgaaatatgaaatgcagctattggatgaactcccaatgatatacagctgttgcatatttgtgtactgcatgtttgaatgtttcaagatcaagaactcagtaaactaccatctgctttttaccttagttctattcagtttaatagtaaccacagtttaccttaaggtaaaagagccgatattccatcaggtcatgtatggaatgttggtctttacattagtacttcgatctatttatattgttacatgggtttatccatggcttagaggactgggttatacatcattgggtatatttttattgggatttttattttggaatatagataacatattttgtgagtcactgaggaactttcgaaagaaggtaccacctatcataggtattaccacacaatttcatgcatggtggcatattttaactggccttggttcctatcttcacatccttttcagtttgtatacaagaacactttacctgagatataggccaaaagtgaagtttctctttggaatctggccagtgatcctgtttgagcctctcaggaagcattga

SEQ ID NO: 17 - DNA, 519 bp, Artificial Sequence
  synthetic oligomer
atgatatacagctgttgcatatttgtgtactgcatgtttgaatgtttcaagatcaagaactcagtaaactaccatctgctttttaccttagttctattcagtttaatagtaaccacagtttaccttaaggtaaaagagccgatattccatcaggtcatgtatggaatgttggtctttacattagtacttcgatctatttatattgttacatgggtttatccatggcttagaggactgggttatacatcattgggtatatttttattgggatttttattttggaatatagataacatattttgtgagtcactgaggaactttcgaaagaaggtaccacctatcataggtattaccacacaatttcatgcatggtggcatattttaactggccttggttcctatcttcacatccttttcagtttgtatacaagaacactttacctgagatataggccaaaagtgaagtttctctttggaatctggccagtgatcctgtttgagcctctcaggaagcattga

SEQ ID NO: 18 - DNA, 1965 bp, Artificial Sequence
  synthetic oligomer
atgaatggacaccttgaagcagaggagcagcaggaccagaggccagaccaggagctgaccgggagctggggccacgggcctaggagcaccctggtcagggctaaggccatggccccgcccccaccgccactggctgccagcaccccgctcctccatggcgagtttggctcctacccagcccgaggcccacgctttgccctcacccttacatcgcaggccctgcacatacagcggctgcgccccaaacctgaagccaggccccggggtggcctggtcccgttggccgaggtctcaggctgctgcaccctgcgaagccgcagcccctcagactcagcggcctacttctgcatctacacctaccctcggggccggcgcggggcccggcgcagagccactcgcaccttccgggcagatggggccgccacctacgaagagaaccgtgccgaggcccagcgctgggccactgccctcacctgtctgctccgaggactgccactgcccggggatggggagatcacccctgacctgctacctcggccgccccggttgcttctattggtcaatccctttgggggtcggggcctggcctggcagtggtgtaagaaccacgtgcttcccatgatctctgaagctgggctgtccttcaacctcatccagacagaacgacagaaccacgcccgggagctggtccaggggctgagcctgagtgagtgggatggcatcgtcacggtctcgggagacgggctgctccatgaggtgctgaacgggctcctagatcgccctgactgggaggaagctgtgaagatgcctgtgggcatcctcccctgcggctcgggcaacgcgctggccggagcagtgaaccagcacgggggatttgagccagccctgggcctcgacctgttgctcaactgctcactgttgctgtgccggggtggtggccacccactggacctgctctccgtgacgctggcctcgggctcccgctgtttctccttcctgtctgtggcctggggcttcgtgtcagatgtggatatccagagcgagcgcttcagggccttgggcagtgcccgcttcacactgggcacggtgctgggcctcgccacactgcacacctaccgcggacgcctctcctacctccccgccactgtggaacctgcctcgcccacccctgcccatagcctgcctcgtgccaagtcggagctgaccctaaccccagacccagccccgcccatggcccactcacccctgcatcgttctgtgtctgacctgcctcttcccctgccccagcctgccctggcctctcctggctcgccagaacccctgcccatcctgtccctcaacggtgggggcccagagctggctggggactggggtggggctggggatgctccgctgtccccggacccactgctgtcttcacctcctggctctcccaaggcagctctacactcacccgtctccgaaggggcccccgtaattcccccatcctctgggctcccacttcccacccctgatgcccgggtaggggcctccacctgcggcccgcccgaccacctgctgcctccgctgggcaccccgctgcccccagactgggtgacgctggagggggactttgtgctcatgttggccatctcgcccagccacctaggcgctgacctggtggcagctccgcatgcgcgcttcgacgacggcctggtgcacctgtgctgggtgcgtagcggcatctcgcgggctgcgctgctgcgccttttcttggccatggagcgtggtagccacttcagcctgggctgtccgcagctgggctacgccgcggcccgtgccttccgcctagagccgctcacaccacgcggcgtgctcacagtggacggggagcaggtggagtatgggccgctacaggcacagatgcaccctggcatcggtacactgctcactgggcctcctggctgcccggggcgggagccctga

SEQ ID NO: 19 - DNA, 1614 bp, Artificial Sequence
  synthetic oligomer
atgggggcgacgggggcggcggagccgctgcaatccgtgctgtgggtgaagcagcagcgctgcgccgtgagcctggagcccgcgcgggctctgctgcgctggtggcggagcccggggcccggagccggcgcccccggcgcggatgcctgctctgtgcctgtatctgagatcatcgccgttgaggaaacagacgttcacgggaaacatcaaggcagtggaaaatggcagaaaatggaaaagccttacgcttttacagttcactgtgtaaagagagcacgacggcaccgctggaagtgggcgcaggtgactttctggtgtccagaggagcagctgtgtcacttgtggctgcagaccctgcgggagatgctggagaagctgacgtccagaccaaagcatttactggtatttatcaacccgtttggaggaaaaggacaaggcaagcggatatatgaaagaaaagtggcaccactgttcaccttagcctccatcaccactgacatcatcgttactgaacatgctaatcaggccaaggagactctgtatgagattaacatagacaaatacgacggcatcgtctgtgtcggcggagatggtatgttcagcgaggtgctgcacggtctgattgggaggacgcagaggagcgccggggtcgaccagaaccacccccgggctgtgctggtccccagtagcctccggattggaatcattcccgcagggtcaacggactgcgtgtgttactccaccgtgggcaccagcgacgcagaaacctcggcgctgcatatcgttgttggggactcgctggccatggatgtgtcctcagtccaccacaacagcacactccttcgctactccgtgtccctgctgggctacggcttctacggggacatcatcaaggacagtgagaagaaacggtggttgggtcttgccagatacgacttttcaggtttaaagaccttcctctcccaccactgctatgaagggacagtgtccttcctccctgcacaacacacggtgggatctccaagggataggaagccctgccgggcaggatgctttgtttgcaggcaaagcaagcagcagctggaggaggagcagaagaaagcactgtatggtttggaagctgcggaggacgtggaggagtggcaagtcgtctgtgggaagtttctggccatcaatgccacaaacatgtcctgtgcttgtcgccggagccccaggggcctctccccggctgcccacttgggagacgggtcttctgacctcatcctcatccggaaatgctccaggttcaattttctgagatttctcatcaggcacaccaaccagcaggaccagtttgacttcacttttgttgaagtttatcgcgtcaagaaattccagtttacgtcgaagcacatggaggatgaggacagcgacctcaaggagggggggaagaagcgctttgggcacatttgcagcagccacccctcctgctgctgcaccgtctccaacagctcctggaactgcgacggggaggtcctgcacagccctgccatcgaggtcagagtccactgccagctggttcgactctttgcacgaggaattgaagagaatccgaagccagactcacacagctga

SEQ ID NO: 20 - DNA, 4779 bp, Artificial Sequence
  synthetic oligomer
  misc_feature (980)..(982): n is a, c, g, or t
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcnnnggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 21 - DNA, 22 bp, Artificial Sequence
  synthetic oligo
acaggattcaaaccaggactgt

SEQ ID NO: 22 - DNA, 20 bp, Artificial Sequence
  synthetic oligo
tgggcatctttccttccgaa

SEQ ID NO: 23 - DNA, 22 bp, Artificial Sequence
  synthetic oligo
tgacaggattcaaaccaggact

SEQ ID NO: 24 - DNA, 20 bp, Artificial Sequence
  synthetic oligo
ctgggcatctttccttccga

SEQ ID NO: 25 - DNA, 22 bp, Artificial Sequence
  synthetic oligo
atactcaccgaacggaagaacc

SEQ ID NO: 26 - DNA, 22 bp, Artificial Sequence
  synthetic oligo
ccattagcccattcaccacctc

SEQ ID NO: 27 - DNA, 21 bp, Artificial Sequence
  synthetic oligo
actgatactcaccgaacggaa

SEQ ID NO: 28 - DNA, 21 bp, Artificial Sequence
  synthetic oligo
cattagcccattcaccacctc

SEQ ID NO: 29 - DNA, 20 bp, Artificial Sequence
  synthetic oligo
cacagccaacagtctccaaa

SEQ ID NO: 30 - DNA, 19 bp, Artificial Sequence
  synthetic oligo
tctgagtataagccgccca

SEQ ID NO: 31 - DNA, 19 bp, Artificial Sequence
  synthetic oligo
atagaccgagcacagccaa

SEQ ID NO: 32 - DNA, 21 bp, Artificial Sequence
  synthetic oligo
gaaccttctcaggattgaggt

SEQ ID NO: 33 - DNA, 21 bp, Artificial Sequence
  synthetic oligo
taacgaacgagactctggcat

SEQ ID NO: 34 - DNA, 22 bp, Artificial Sequence
  synthetic oligo
cggacatctaagggcatcacag